(19) United States
(12) Patent Application Publication
(10) Pub. No.: US 2009/0022709 A1
(43) Pub. Date: Jan. 22, 2009

(54) Control of sorbose utilization genes and uses thereof

(76) Inventors: Elena Rustchenko Bulgac, Rochester, NY (US); M. Anaul Kabir, Karanataka (IN)

Correspondence Address: Ballard Spahr Andrews & Ingersoll, LLP, SUITE 1000, 999 PEACHTREE STREET, ATLANTA, GA, 30309-3915, US

(21) Appl. No.: 11/997,707

(22) PCT Filed: Aug. 2, 2006
(86) PCT No.: PCT/US06/30057

§ 371 Date: Oct. 3, 2008

Related U.S. Application Data
(60) Provisional application No. 60/704,853, filed on Aug. 2, 2005.

(51) Int. Cl. A61K 39/395; C07K 14/00; C12N 15/11; C12N 1/19; C07K 16/18; A61P 31/00; C12Q 1/68; G01N 33/53; A61K 31/7088

(52) U.S. Cl. 424/130.1; 530/324; 536/23.1; 435/254.22; 530/387.9; 435/6; 536/24.32; 435/7.31; 514/44; 530/350

(57) ABSTRACT

This invention relates generally to the discovery of a novel control of sorbose utilization (CSU) genes. The invention further relates to the detection of these genes and their use in the diagnosis and treatment of fungal infections.

This application claims priority to U.S. Provisional Application Ser. No. 60/704,853, filed Aug. 2, 2005 which is hereby incorporated in its entirety by this reference.

### FIELD OF THE INVENTION

This invention relates generally to the discovery of a novel control of sorbose utilization (CSU) genes. The invention further relates to the detection of these genes, and/or CSU51 expression in the diagnosis and treatment of fungal infections.

### SUMMARY OF THE INVENTION

The present invention provides purified CSU polypeptides and nucleic acids encoding these polypeptides. Also provided by the present invention are antibodies that specifically bind to CSU polypeptides.

Also provided by the present invention is a method for detecting the presence of a CSU nucleic acid or CSU polypeptide in a sample.

Further provided is a cell with a mutated or altered CSU nucleic acid.

The present invention also provides methods of reducing expression of a CSU nucleic acid or polypeptide in a cell.

The present invention also provides a method of identifying an agent that modulates an activity of a CSU gene comprising the steps of: a) contacting a host cell comprising a polynucleotide sequence that encodes a functional CSU polypeptide with a test agent; and b) determining if at least one activity of a CSU polypeptide is modulated, such that if an activity of a CSU polypeptide is modulated, the test agent is an agent that modulates CSU activity.

Also provided is a method of treating a fungal infection by administering an agent that modulates an activity of a CSU polypeptide or nucleic acid.

### BACKGROUND

Candida is a yeast and the most common cause of opportunistic mycoses worldwide. It is also a frequent colonizer of human skin and mucous membranes. Candida is a member of normal flora of skin, mouth, vagina, and stool. As well as being a pathogen and a colonizer, it is found in the environment, particularly on leaves, flowers, water, and soil. While most of the Candida spp. are mitosporic, some have known teleomorphic state and produce sexual spores.

The genus Candida includes around 154 species. Among these, six are most frequently isolated in human infections. While Candida albicans is the most abundant and significant species, Candida tropicalis, Candida glabrata, Candidaparapsilosis, Candida krusei, and Candida lusitaniae are also isolated as causative agents of Candida infections.

Importantly, there has been a recent increase in infections due to non-albicans Candida spp., such as Candida glabrata and Candida krusei. Patients receiving fluconazole prophylaxis are particularly at risk of developing infections due to fluconazole-resistant Candida krusei and Candida glabrata strains. Nevertheless, the diversity of Candida spp. that are encountered in infections is expanding and the emergence of other species that were rarely in play in the past is now likely. Infections caused by Candida spp. are in general referred to as candidiasis. The clinical spectrum of candidiasis is extremely diverse. Almost any organ or system in the body can be affected. Candidiasis may be superficial and local or deep-seated and disseminated. Disseminated infections arise from hematogenous spread from the primarily infected locus. Candida albicans is the most pathogenic and most commonly encountered species among all. Its ability to adhere to host tissues, produce secretory aspartyl proteases and phospholipase enzymes, and transform from yeast to hyphal phase are the major determinants of its pathogenicity. Several host factors predispose to candidiasis. Candidiasis is mostly an endogenous infection, arising from overgrowth of the fungus inhabiting in the normal flora. However, it may occasionally be acquired from exogenous sources (such as catheters or prosthetic devices) or by person-to-person transmission (such as oral candidiasis in neonates of mothers with vaginal candidiasis or endophthalmitis following corneal transplantation from an infected donor).

Current therapy available for systemic candidiasis is limited to the use of anti-fungal agents. In practice, current anti-fungal therapy is based on a few antimycotics, such as flucytosine, amphotericin B and azole derivatives. Many of these antimycotics are somewhat water insoluble, which restrict their bioavailability and present problems in intravenous formulation. In addition, they cause serious and often difficult side effects, such as renal toxicity, bone marrow destruction, as well as unpleasant symptoms such as fever and shivering. Furthermore, the chronic use of these anti-fungal agents has led to the emergence of drug-resistant strains of Candida, which can cause fatal relapse of the disease. In view of the alarming prevalence of life-threatening candidiasis and the lack of satisfactory agents to treat this condition, there is a pressing need for developing better therapeutic agents to combat C. albicans infections.

### BRIEF DESCRIPTION OF THE FIGURES

FIG. 1 shows determining fragments 135 and 140 within the deletion of the Sou⁺ mutant Sor5. (A) The three short chromosomes, indicated with a bracket, of the strain 3153A and its mutant Sor5 as separated with OFAGE apparatus. The remaining chromosomes are clustered together at the top of the gel. The bands for the chromosomes 5a, 5b, and 5a-Δ are indicated with arrows. Note that the 3153A has homologue 5b shorter due to a deletion (6). (B) Signals obtained with the filter corresponding to the gel in (A), and with the probe prepared from the chromosome fragment 135. (C) Schematic representation of the chromosomes 5a, 5b, and 5a-Δ, which are shown in (A). A single chromosome is indicated with a dotted line. Two co-migrating chromosomes, 6b and 7a, are indicated with a continuous line.

FIG. 2 shows the five functional Regions 140 (encompassing A and B), 135, C, and 139 that contain CSU51 and CSU52-CSU5S genes. (A) The schematic representation of chromosome 5 including contigs in the right arm as indicated with the open bars designated 1-6 (16). See Table 4 for the contig nomenclature. The tentative position but not the alignment of contigs 5 and 6 have been determined. The gap between contigs 4 and 3 has been sequenced, and the sizes of two other gaps between contigs 3 and 2 and contigs 2 and 1 were deduced. The approximate positions of the SfiI site, CAG1, ADH1, and HIS1 genes, as well as cloned fragments 133, 134, 137, 138, 141, and 142 that are distributed along chromosome 5 outside the contigs 1-6 are indicated. The following are indicated in parenthesis as kbp: chromosome 5 length; a total length of contigs 1-4 including gaps; a suggested deletion in S or S mutant. (B) The hemizygous truncation of chromosome 5 at sites 7-12, but not at sites 3-6, conferred the Sou⁺ phenotype. The positions of the fragmentation sites 1-12, as well as the associated Sou phenotypes are presented above contigs 1, 3, and 4. (C) The five functional Regions presented as filled boxes with their positions and sizes relative to contigs and fragmentation sites in (B). (D) The large Deletion VII of approximately 78.5 kbp on one chromosome 5, which is presented in scale with (B) and (C), does not change the Sou⁻ phenotype. (E) The four types of hemizygous constructs that conferred the Sou⁺ phenotype: 1, 140-Δ 135-Δ; 2, 140-Δ (by Deletion VII) C-Δ 139-Δ (by fragmentation 3); 3, A-Δ C-Δ 139-Δ (by fragmentation 3); 4, csu51-Δ C-Δ 139-Δ (by fragmentation 3). The internal deletions and truncations are presented in scale with (B-D). (F) Identification of CSU51 in the 953 bp sequence of Region A. The fragmentation sites 6-8, within and flanking Region A, are shown as boxed numbers with the corresponding Sou phenotypes. The nucleotide positions of the five ORFs are indicated. Asterisks designate the approximate positions of the frameshift mutations that were introduced in each ORF by site-directed mutagenesis. (G) The Sou phenotype of five strains transformed with pCA88-derived plasmids that co-express SOU1 along with differently mutated Region A as described above. Each of orf1-orf5 designates a mutated ORF in Region A. Two control strains that carry either pCA88 with SOU1 alone (pCA88) or the normal sequence of Region A (Reg. A), are also shown. Note that only destruction of ORF3 [circled asterisk in (F)] allowed expression of SOU1, leading to a Sou⁺ phenotype on L-sorbose plate.

FIG. 3 shows the upstream region of CSU51 (ORF3), ORF, Csu51p, and 3′-UTR of CSU51. The nucleotides are numbered by assigning A of the ATG initiator codon as position 1. Putative TATA boxes located at positions −12, −146, −446 and −461.

FIG. 4 shows the method of concomitant deletion of one copy of ORF3 (CSU51) and one chromosome fragmentation.

### DETAILED DESCRIPTION OF THE INVENTION

Candida albicans is an important opportunistic fungal pathogen that is normally found in healthy humans. C. albicans is a natural diploid containing eight pairs of chromosomes, which does not have mating between haploids, but can perform a parasexual cycle in vitro, starting from mating the diploids and finishing with the tetraploids randomly losing chromosomes and subsequently returning to the diploids (1). It was established that C. albicans population gains genetic variability in vitro due to the high frequency chromosome instability (reviewed in 2). Most unusual, however, was the finding that C. albicans uses specific alterations of different specific chromosomes to survive and adapt to different adverse environments. Among specific alterations, reversible change of chromosome copy number is a prominent, albeit, an unusual means of regulation. For example, utilization of the secondary carbon sources L-sorbose (Sou⁺) and D-arabinose (Aru⁺) depends, respectively, on the chromosome 5 monosomy and chromosome 6 monosomy or, alternatively, chromosome 2 trisomy. Primary resistance to the antibiotic fluconazole (Flu^{R}) and 5-fluoro-orotic acid ((Foa^{R}) depends on chromosome 4 monosomy and chromosome 4 trisomy, respectively. Control of sorbose utilization was studied in detail because it involves the loss of only chromosome 5 that can be reversed by duplication of the remaining homologue (2-8). The growth on sorbose depends on the SOU1 gene that encodes NADPH-dependent sorbose reductase, which converts L-sorbose to D-sorbitol leading to fructose. The loss of one copy of chromosome 5 up-regulates SOU1, which resides on another chromosome. During Sou⁻ to Sou⁺ transition, the SOU1 transcript increases several fold, although both SOUL copies and its upstream sequences remain intact, which shows that copy number of chromosome 5 controls the copy number of a CSU gene (Control of Sorbose Utilization) encoding a negative regulator residing on this chromosome. The ratio between CSU and SOU1, thus, determines the Sou phenotype. (3, 8-10).

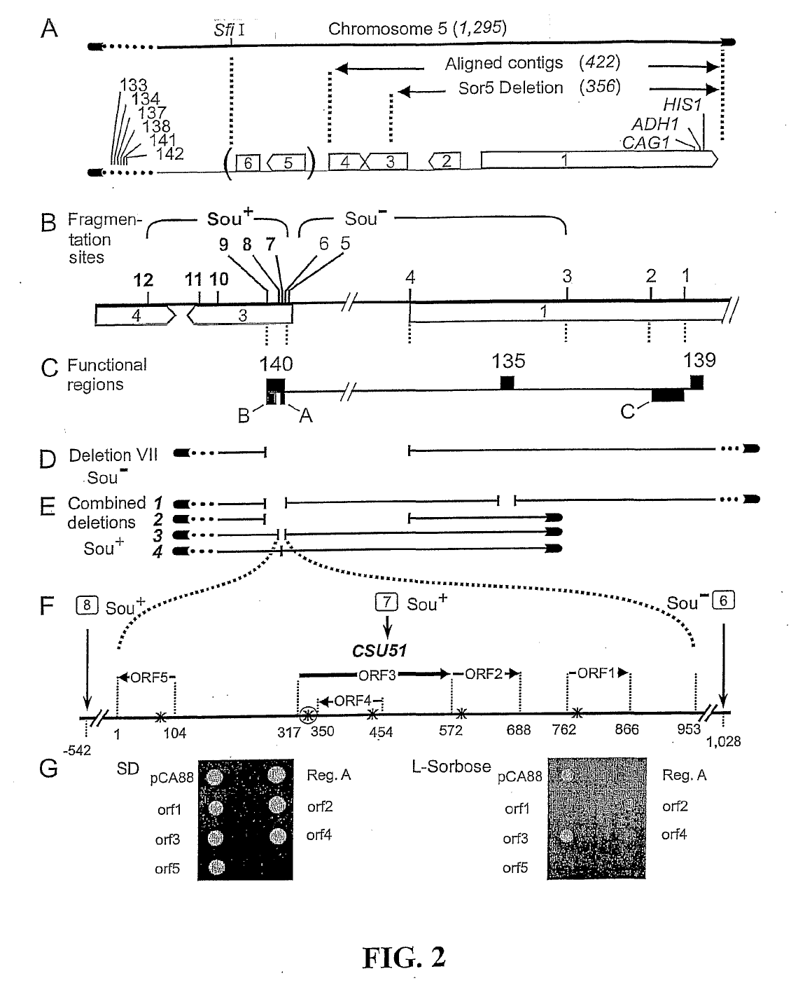

As set forth herein, the present invention shows that chromosome 5 of Candida albicans contains at least five functionally, but not structurally, redundant negative regulators, CSU51-CSU55, in five spatially separated regions within an approximately 209 kbp segment (See FIG. 2). Thus, Applicants have identified several negative regulators on this 209 kbp segment of chromosome 5. One of these is a gene located in this 209 kbp segment, CSU51, which encodes a novel protein of the helix-loop-helix (HLH) type. Other novel genes identified in this region, and set forth herein, include CSU52, CSU53 and CSU55. The loss of one copy of all five regions or some of them in particular combinations, allowed the growth on a sorbose plate, which mimics the Sou⁺ phenotype due to the loss of one chromosome 5. Multiple redundant regulators explain in a simple, elegant way why the entire chromosome needs to be lost in Sou⁺ mutants.

In addition to participating in regulation of the sorbose utilization pathway, CSU51, is a novel gene that is found in the Candida albicans genome, but is not found in any mammalian genome. Therefore, this gene and its gene product(s) provide specific targets for anti-fungal agents that treat a Candida infection with reduced interaction, if any, with human genes or human gene products, thus minimizing the side effects associated with current antifungal therapy.

Other targets include the genes and gene products involved in the CSU51 pathway and also any other CSU genes and CSU gene products involved in a functionally redundant pathway(s). For example, the present invention shows that CSU51, CSU52, CSU53 and CSU55 are involved in a redundant negative regulation pathway located on chromosome 5 [for example, in sequence 140 (encompassing regions A and B), region 135, region C and region 139]. Therefore, negative regulators (CSU52-55) found in these regions and their gene products are also set forth herein as targets for antifungal therapy. Thus, CSU51 can be targeted alone or in combination with one or more additional negative regulators, such as CSU52, CSU53 and CSU55, found on chromosome 5. The genes regulated by CSU51 either alone or in combination with other negative regulators found on chromosome 5 are also set forth herein as targets for antifungal therapy.

CSU51, a helix-loop-helix protein, is a transcription factor that binds to the SOU1 promoter to prevent efficient transcription of this gene, or forms heterodimers with other transcription factors to regulate SOU1. In addition to providing a target for antifungal therapy, the discovery of CSU51 and its involvement in the mechanism of chromosome loss, could explain other phenomena such as the development of cancers via loss of chromosomes containing genes involved in negative regulation of genes. CSU52, CSU53 and CSU55 can also function as transcription factors that bind to the SOU1 promoter or form heterodimers with other transcription factors that regulate SOU1.

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific nucleic acids, specific polypeptides, or to particular methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “an antibody” includes mixtures of antibodies, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally obtained prior to treatment” means obtained before treatment, after treatment, or not at all.

As used throughout, by “subject” is meant an individual. Preferably, the subject is a mammal such as a primate, and, more preferably, a human. The term “subject” includes domesticated animals, such as cats, dogs, etc., fish, reptiles, birds, livestock (e.g., cattle, horses, pigs, sheep, goats, etc.), and laboratory animals (e.g., mouse, rabbit, rat, gerbil, guinea pig, etc.).

### CSU Polypeptides

The present invention provides isolated CSU polypeptides. The CSU polypeptide of the present invention can be a CSU51 polypeptide, a CSU52 polypeptide, a CSU53 polypeptide, a CSU55 polypeptide or a fragment of any of these polypeptides. Further provided by the present invention is an isolated polypeptide comprising SEQ ID NO: 1. SEQ ID NO: 1 corresponds to a full-length CSU51 polypeptide of 84 amino acids found in Candida albicans. The present invention also provides fragments of CSU51 polypeptides, for example, fragments of SEQ ID NO: 1. These fragments can be of sufficient length to serve as antigenic peptides for the generation of anti-CSU51 antibodies. The present invention also contemplates functional fragments of CSU51 that possess at least one activity of CSU51, such as, the ability to repress or inhibit SOU1 expression or the ability to repress or inhibit Candida albicans sorbose utilization. It is understood that a CSU51 polypeptide or a fragment thereof may exist in more than one form, such as a single CSU51 polypeptide, an assembly of at least one CSU51 polypeptide, and/or within a complex (i.e., comprising multi-subunits) containing at least one CSU51 polypeptide with at least one other polypeptide. For example, a CSU51 polypeptide or fragment thereof of the present invention can exist as a complex with a transcription factor or as a complex with a nucleic acid such as a promoter region for a gene under the transcriptional control of CSU51. Fragments and variants of CSU51 can include one or more conservative amino acid residues as compared to the amino acid sequence of SEQ ID NO: 1.

The present invention also provides an isolated polypeptide comprising SEQ ID NO: 6. SEQ ID NO: 6 corresponds to a full-length CSU52 polypeptide found in Candida albicans. The present invention also provides fragments of CSU52 polypeptides, for example, fragments of SEQ ID NO: 6. These fragments can be of sufficient length to serve as antigenic peptides for the generation of anti-CSU52 antibodies. The present invention also contemplates functional fragments of CSU52 that possess at least one activity of CSU52, such as, the ability to repress or inhibit SOU1 expression or the ability to repress or inhibit Candida albicans sorbose utilization. It is understood that a CSU52 polypeptide or a fragment thereof may exist in more than one form, such as a single CSU52 polypeptide, an assembly of at least one CSU52 polypeptide, and/or within a complex (i.e., comprising multi-subunits) containing at least one CSU52 polypeptide with at least one other polypeptide. For example, a CSU52 polypeptide or fragment thereof of the present invention can exist as a complex with a transcription factor or as a complex with a nucleic acid such as a promoter region for a gene under the transcriptional control of CSU52. Fragments and variants of CSU52 can include one or more conservative amino acid residues as compared to the amino acid sequence of SEQ ID NO: 6.

Further provided is an isolated polypeptide comprising SEQ ID NO: 7. SEQ ID NO: 7 corresponds to a full-length CSU53 polypeptide found in Candida albicans. The present invention also provides fragments of CSU53 polypeptides, for example, fragments of SEQ ID NO: 7. These fragments can be of sufficient length to serve as antigenic peptides for the generation of anti-CSU53 antibodies. The present invention also contemplates functional fragments of CSU53 that possess at least one activity of CSU53, such as, the ability to repress or inhibit SOU1 expression or the ability to repress or inhibit Candida albicans sorbose utilization. It is understood that a CSU53 polypeptide or a fragment thereof may exist in more than one form, such as a single CSU53 polypeptide, an assembly of at least one CSU53 polypeptide, and/or within a complex (i.e., comprising multi-subunits) containing at least one CSU53 polypeptide with at least one other polypeptide. For example, a CSU53 polypeptide or fragment thereof of the present invention can exist as a complex with a transcription factor or as a complex with a nucleic acid such as a promoter region for a gene under the transcriptional control of CSU53. Fragments and variants of CSU53 can include one or more conservative amino acid residues as compared to the amino acid sequence of SEQ ID NO: 7.

Also provided is an isolated polypeptide comprising SEQ ID NO: 8. SEQ ID NO: 8 corresponds to a full-length CSU55 polypeptide found in Candida albicans. The present invention also provides fragments of CSU55 polypeptides, for example, fragments of SEQ ID NO: 8. These fragments can be of sufficient length to serve as antigenic peptides for the generation of anti-CSU55 antibodies. The present invention also contemplates functional fragments of CSU55 that possess at least one activity of CSU55, such as, the ability to repress or inhibit SOU1 expression or the ability to repress or inhibit Candida albicans sorbose utilization. It is understood that a CSU55 polypeptide or a fragment thereof may exist in more than one form, such as a single CSU55 polypeptide, an assembly of at least one CSU55 polypeptide, and/or within a complex (i.e., comprising multi-subunits) containing at least one CSU55 polypeptide with at least one other polypeptide. For example, a CSU55 polypeptide or fragment thereof of the present invention can exist as a complex with a transcription factor or as a complex with a nucleic acid such as a promoter region for a gene under the transcriptional control of CSU55. Fragments and variants of CSU55 can include one or more conservative amino acid residues as compared to the amino acid sequence of SEQ ID NO: 8.

By “isolated polypeptide” or “purified polypeptide” is meant a polypeptide that is substantially free from the materials with which the polypeptide is normally associated in nature or in culture. The polypeptides of the invention can be obtained, for example, by extraction from a natural source if available (for example, a mammalian cell), by expression of a recombinant nucleic acid encoding the polypeptide (for example, in a cell or in a cell-free translation system), or by chemically synthesizing the polypeptide. In addition, polypeptide may be obtained by cleaving full length polypeptides. When the polypeptide is a fragment of a larger naturally occurring polypeptide, the isolated polypeptide is shorter than and excludes the full-length, naturally-occurring polypeptide of which it is a fragment.

The polypeptides of the invention can be prepared using any of a number of chemical polypeptide synthesis techniques well known to those of ordinary skill in the art including solution methods and solid phase methods. One method of producing the polypeptides of the present invention is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to a CSU polypeptide of the present invention, for example, can be synthesized by standard chemical reactions (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY). Alternatively, the peptide or polypeptide is independently synthesized in vivo as described above.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

The polypeptides of the invention can also be prepared by other means including, for example, recombinant techniques. Examples of appropriate cloning and sequencing techniques, and instructions sufficient to direct persons of skill through many cloning exercises are found in Sambrook et al. (2001) Molecular Cloning—A Laboratory Manual (3rd ed.) Vol. 1-3, Cold Spring Harbor Laboratory, Cold Spring Harbor Press, NY, (Sambrook).

Also provided by the present invention is a polypeptide comprising an amino acid sequence at least about 50%, 55%, 60%, 65%, 70%, 75%, 80%, 85%, 90%, 95%, 96%, 97%, 98% or 99% identical to SEQ ID NO: 1, SEQ ID NO: 6, SEQ ID NO: 7 or SEQ ID NO: 8. A polypeptide comprising an amino acid sequence at least 50%, 55%, 60%, 65%, 70%, 75%, 80%, 85%, 90%, 95%, 96%, 97%, 98% or 99% identical to a fragment of SEQ ID NO: 1, SEQ ID NO: 6, SEQ ID NO: 7 or SEQ ID NO: 8 is also provided. The present invention also provides homologs of CSU51, CSU52, CSU53 and CSU55. For example, the present invention provides a CSU51 polypeptide comprising an amino acid sequence that is at least about 50%, 55%, 60%, 65%, 70%, 75%, 80%, 85%, 90%, 95%, 96%, 97%, 98% or 99% identical to SEQ ID NO: 1 or fragments of SEQ ID NO: 1 and is identified or isolated from other fungal species, for example a Candida species such as, but not limited to, Candida albicans, Candida tropicalis, Candida glabrata, Candida parapsilosis, Candida krusei, Candida lusitaniae, Candida kefyr, Candida guilliermondii, Candida dubliniensis, Candida ciferri, Candida famata, Candida lambica, Candida lipolytica, Candida norvegensis, Candida rugosa, Candida viswanathii and Candida zeylanoid. Similarly, the present invention provides a polypeptide that is at least 50%, 55%, 60%, 65%, 70%, 75%, 80%, 85%, 90%, 95%, 96%, 97%, 98% or 99% identical to SEQ ID NO: 6 (CSU52) or a fragment thereof, SEQ ID NO: 7 (CSU53) or a fragment thereof, SEQ ID NO: 8 (CSU55) or a fragment thereof, and is identified or isolated from other fungal species. Therefore, as utilized herein, the CSU51 polypeptides of the present invention include homologs of the Candida albicans polypeptide set forth herein as SEQ ID NO: 1. Similarly, the CSU52 polypeptides of the present invention include homologs of the Candida albicans polypeptide set forth herein as SEQ ID NO: 6. Furthermore, the CSU53 polypeptides of the present invention include homologs of the Candida albicans polypeptide set forth herein as SEQ ID NO: 7 and the CSU55 polypeptides of the present invention include homologs of the Candida albicans polypeptide set forth herein as SEQ ID NO: 8.

It is understood that as discussed herein the use of the terms “homology” and “identity” mean the same thing as similarity. Thus, for example, if the use of the word homology is used to refer to two non-natural sequences, it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of homology to specific known sequences. In general, variants of nucleic acids and polypeptides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two polypeptides or nucleic acids. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/b12seq/b12.html)), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Also provided by the present invention is a polypeptide comprising SEQ ID NO: 1, SEQ ID NO: 6, SEQ ID NO: 7 or SEQ ID NO: 8 with one or more conservative amino acid substitutions. These conservative substitutions are such that a naturally occurring amino acid is replaced by one having similar properties. Such conservative substitutions do not alter the function of the polypeptide. For example, conservative substitutions can be made according to the following table:

Amino Acid Substitutions

| Original Residue | Exemplary Substitutions |
|---|---|
| Arg | Lys |
| Asn | Gln |
| Asp | Glu |
| Cys | Ser |
| Gln | Asn |
| Glu | Asp |
| Gly | Pro |
| His | Gln |
| Ile | leu; val |
| Leu | ile; val |
| Lys | arg; gln |
| Met | leu; ile |
| Phe | met; leu; tyr |
| Ser | Thr |
| Thr | Ser |
| Trp | Tyr |
| Tyr | trp; phe |
| Val | ile; leu |

Thus, it is understood that, where desired, modifications and changes may be made in the nucleic acid encoding the polypeptides of this invention and/or amino acid sequence of the polypeptides of the present invention and still obtain a polypeptide having like or otherwise desirable characteristics. Such changes may occur in natural isolates or may be synthetically introduced using site-specific mutagenesis, the procedures for which, such as mis-match polymerase chain reaction (PCR), are well known in the art. For example, certain amino acids may be substituted for other amino acids in a polypeptide without appreciable loss of functional activity. It is thus contemplated that various changes may be made in the amino acid sequence of the polypeptides of the present invention (or underlying nucleic acid sequence) without appreciable loss of biological utility or activity and possibly with an increase in such utility or activity.

### Nucleic Acids

The present invention also provides nucleic acids that encode CSU polypeptides and variants or fragments thereof. The present invention also provides an isolated nucleic acid encoding a polypeptide comprising SEQ ID NO: 1, SEQ ID NO: 6, SEQ ID NO: 7 or SEQ ID NO: 8. Nucleic acids encoding fragments of SEQ ID NO: 1, SEQ ID NO: 6, SEQ ID NO: 7 or SEQ ID NO: 8 are also provided. The present invention also provides a nucleic acid that encodes an amino acid sequence that is at least about 50%, 55%, 60%, 65%, 70%, 75%, 80%, 85%, 90%, 95%, 96%, 97%, 98% or 99% identical to SEQ ID NO: 1 or fragments of SEQ ID NO: 1 and is identified or isolated from other fungal species, for example a Candida species such as, but not limited to, Candida albicans, Candida tropicalis, Candida glabrata, Candida parapsilosis, Candida krusei, Candida lusitaniae, Candida kefyr, Candida guilliermondii, Candida dubliniensis, Candida ciferri, Candida famata, Candida lambica, Candida lipolytica, Candida norvegensis, Candida rugosa, Candida viswanathii and Candida zeylanoide.

The present invention also provides a nucleic acid that encodes an amino acid sequence that is at least about 50%, 55%, 60%, 65%, 70%, 75%, 80%, 85%, 90%, 95%, 96%, 97%, 98% or 99% identical to SEQ ID NO: 6 or fragments of SEQ ID NO: 6 and is identified or isolated from other fungal species, for example a Candida species such as, but not limited to, Candida albicans, Candida tropicalis, Candida glabrata, Candida parapsilosis, Candida krusei, Candida lusitaniae, Candida kefyr, Candida guilliermondii, Candida dubliniensis, Candida ciferri, Candida famata, Candida lambica, Candida lipolytica, Candida norvegensis, Candida rugosa, Candida viswanathii and Candida zeylanoide.

The present invention also provides a nucleic acid that encodes an amino acid sequence that is at least about 50%, 55%, 60%, 65%, 70%, 75%, 80%, 85%, 90%, 95%, 96%, 97%, 98% or 99% identical to SEQ ID NO: 7 or fragments of SEQ ID NO: 7 and is identified or isolated from other fungal species, for example a Candida species such as, but not limited to, Candida albicans, Candida tropicalis, Candida glabrata, Candida parapsilosis, Candida krusei, Candida lusitaniae, Candida kefyr, Candida guilliermondii, Candida dubliniensis, Candida ciferri, Candida famata, Candida lambica, Candida lipolytica, Candida norvegensis, Candida rugosa, Candida viswanathii and Candida zeylanoide.

The present invention also provides a nucleic acid that encodes an amino acid sequence that is at least about 50%, 55%, 60%, 65%, 70%, 75%, 80%, 85%, 90%, 95%, 96%, 97%, 98% or 99% identical to SEQ ID NO: 8 or fragments of SEQ ID NO: 8 and is identified or isolated from other fungal species, for example a Candida species such as, but not limited to, Candida albicans, Candida tropicalis, Candida glabrata, Candida parapsilosis, Candida krusei, Candida lusitaniae, Candida kefyr, Candida guilliermondii, Candida dubliniensis, Candida ciferri, Candida famata, Candida lambica, Candida lipolytica, Candida norvegensis, Candida rugosa, Candida viswanathii and Candida zeylanoide.

An example of a nucleic acid encoding a polypeptide comprising SEQ ID NO: 1 is provided herein as SEQ ID NO: 2. SEQ ID NO: 3 also encodes SEQ ID NO:1 and includes the upstream region of CSU51 and the 3′UTR of CSU51. An example of a nucleic acid encoding a polypeptide comprising SEQ ID NO: 6 is provided herein as SEQ ID NO: 9. An example of a nucleic acid encoding a polypeptide comprising SEQ ID NO: 7 is provided herein as SEQ ID NO: 10. An example of a nucleic acid encoding a polypeptide comprising SEQ ID NO: 8 is provided herein as SEQ ID NO: 11.

As used herein, the term “nucleic acid” refers to single or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in Sambrook et al. (2001) Molecular Cloning—A Laboratory Manual (3rd ed.) Vol. 1-3, Cold Spring Harbor Laboratory, Cold Spring Harbor Press, NY, (Sambrook).

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991), which are incorporated herein in their entirety for these methods. These techniques can be used to alter the coding sequence without altering the amino acid sequence that is encoded.

### Vectors, Cells, and Methods of Using

Also provided is a vector, comprising a nucleic acid of the present invention. The vector can direct the in vivo or in vitro synthesis of any of the polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.). The vector, for example, can be a plasmid. The vectors can contain genes conferring hygromycin resistance, gentamicin resistance, or other genes or phenotypes suitable for use as selectable markers, or methotrexate resistance for gene amplification. The vector can comprise the nucleic acid in pET15b, pSRα-Neo, pPICZα, or pPIC9K, or any other vector suitable for the expression of the nucleic acid of the present invention. There are numerous other E. coli (Escherichia coli) expression vectors, known to one of ordinary skill in the art, which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures. Also, nucleic acid modifications can be made to promote amino terminal homogeneity.

Additionally, yeast expression can be used. The invention provides a nucleic acid encoding a polypeptide of the present invention, wherein the nucleic acid can be expressed by a yeast cell. More specifically, the nucleic acid can be expressed by Candida albicans, Pichia pastoris or S. cerevisiae. There are several advantages to yeast expression systems, which include, for example, Saccharomyces cerevisiae and Pichia pastoris. First, evidence exists that proteins produced in a yeast secretion systems exhibit correct disulfide pairing. Second, efficient large scale production can be carried out using yeast expression systems. The Saccharomyces cerevisiae pre-pro-alpha mating factor leader region (encoded by the MFα-1 gene) can be used to direct protein secretion from yeast (Brake, et al.). The leader region of pre-pro-alpha mating factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha mating factor leader region. This construct can be put under the control of a strong transcription promoter, such as the alcohol dehydrogenase I promoter, alcohol oxidase I promoter, a glycolytic promoter, or a promoter for the galactose utilization pathway. The nucleic acid coding sequence is followed by a translation termination codon which is followed by transcription termination signals. Alternatively, the nucleic acid coding sequences can be fused to a second protein coding sequence, such as Sj26 or beta-galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

Mammalian cells permit the expression of proteins in an environment that favors important post-translational modifications such as folding and cysteine pairing, addition of complex carbohydrate structures, and secretion of active protein. Vectors useful for the expression of active proteins in mammalian cells are characterized by insertion of the protein coding sequence between a strong viral promoter and a polyadenylation signal. The vectors can contain genes conferring hygromycin resistance, genticin or G418 resistance, or other genes or phenotypes suitable for use as selectable markers, or methotrexate resistance for gene amplification. The chimeric protein coding sequence can be introduced for example, into a Chinese hamster ovary (CHO) cell line using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other eukaryotic cellular hosts.

Alternative vectors for the expression of genes or nucleic acids in mammalian cells, those similar to those developed for the expression of human gamma-interferon, tissue plasminogen activator, clotting Factor VIII, hepatitis B virus surface antigen, protease Nexinl, and eosinophil major basic protein, can be employed. Further, the vector can include CMV promoter sequences and a polyadenylation signal available for expression of inserted nucleic acids in mammalian cells (such as COS-7).

Insect cells also permit the expression of mammalian proteins. Recombinant proteins produced in insect cells with baculovirus vectors undergo post-translational modifications similar to that of wild-type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild-type occlusion positive viruses. These distinctive plaque morphologies allow visual screening for recombinant viruses in which the AcNPV gene has been replaced with a hybrid gene of choice.

The invention also provides for the vectors containing the contemplated nucleic acids in a host suitable for expressing the nucleic acids. As discussed above, the host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a COS-7 cell, a Pichia cell, a Candida cell (for example from Candida albicans, Candida tropicalis, Candida glabrata, Candidaparapsilosis, Candida krusei, Candida lusitaniae, Candida kefyr, Candida guilliermondii, Candida dubliniensis, Candida ciferri, Candida famata, Candida lambica, Candida lipolytica, Candida norvegensis, Candida rugosa, Candida viswanathii and Candida zeylanoid) or an insect cell.

The present invention provides a method of making any of the CSU polypeptides, fragments and variants described herein comprising: culturing a host cell comprising a vector that encodes a CSU polypeptide, for example, CSU51, CSU52, CSU53 or CSU55, and purifying the polypeptide produced by the host cell. As mentioned above, the CSU51 polypeptides include, but are not limited to, a polypeptide comprising SEQ ID NO:1 or a fragment thereof, polypeptides comprising an amino acid sequence at least about 50% identical to the sequence of SEQ ID NO:1 or a fragment thereof and polypeptides comprising the amino acid sequence of SEQ ID NO:1, or a fragment thereof, with one or more conservative amino acid substitutions. The CSU52 polypeptides include, but are not limited to, a polypeptide comprising SEQ ID NO: 6 or a fragment thereof, polypeptides comprising an amino acid sequence at least about 50% identical to the sequence of SEQ ID NO:6 or a fragment thereof and polypeptides comprising the amino acid sequence of SEQ ID NO:6, or a fragment thereof, with one or more conservative amino acid substitutions. The CSU53 polypeptides include, but are not limited to, a polypeptide comprising SEQ ID NO: 7 or a fragment thereof, polypeptides comprising an amino acid sequence at least about 50% identical to the sequence of SEQ ID NO: 7 or a fragment thereof and polypeptides comprising the amino acid sequence of SEQ ID NO: 7, or a fragment thereof, with one or more conservative amino acid substitutions. The CSU55 polypeptides include, but are not limited to, a polypeptide comprising SEQ ID NO: 8 or a fragment thereof, polypeptides comprising an amino acid sequence at least about 50% identical to the sequence of SEQ ID NO: 8 or a fragment thereof and polypeptides comprising the amino acid sequence of SEQ ID NO: 8, or a fragment thereof, with one or more conservative amino acid substitutions.

### Antibodies

The present invention provides an isolated antibody or fragment thereof that specifically binds to a CSU polypeptide set forth herein, or a fragment thereof. The present invention further provides an isolated antibody or fragment thereof that specifically binds an epitope contained within the amino acid sequence of SEQ ID NO: 1, SEQ ID NO: 6, SEQ ID NO 7 or SEQ ID NO: 8. The present invention also provides an antibody that specifically binds to an epitope contained within the amino acid sequence of a CSU polypeptide of the present invention, for example, within the amino acid sequence of SEQ ID NO: 1, SEQ ID NO: 6, SEQ ID NO 7 or SEQ ID NO: 8, as evidenced by competitive binding studies. Competitive binding studies are well known in the art. For example, if a test antibody competes for binding to the CSU51 polypeptide with an antibody or ligand that specifically binds an epitope contained within amino acids 1-84 of the CSU51 polypeptide, one of skill in the art would readily know that the test antibody binds the same epitope as the antibody or ligand that specifically binds to an epitope contained within or overlapping with amino acids 1-84 of the CSU51 polypeptide.

The antibody of the present invention can be a polyclonal antibody or a monoclonal antibody. The antibody of the invention selectively binds a CSU polypeptide set forth herein. By “selectively binds” or “specifically binds” is meant an antibody binding reaction which is determinative of the presence of the antigen (in the present case, a CSU51, CSU52, CSU53 or CSU55 polypeptide or antigenic fragments thereof) among a heterogeneous population of proteins and other biologics. Thus, under designated immunoassay conditions, the specified antibodies bind preferentially to a particular peptide and do not bind in a significant amount to other proteins in the sample. Specific binding to a CSU polypeptide under such conditions requires an antibody that is selected for its specificity to a CSU polypeptide. Preferably, selective binding includes binding at about or above 1.5 times assay background and the absence of significant binding is less than 1.5 times assay background.

This invention also provides antibodies that compete for binding to natural CSU51 interactors. For example, an antibody of the present invention can compete with CSU51 for binding to another protein or biological molecule, such as a nucleic acid that is under the transcriptional control of CSU51. Further provided are antibodies that compete for binding to natural CSU52 interactors. For example, an antibody of the present invention can compete with CSU52 for binding to another protein or biological molecule, such as a nucleic acid that is under the transcriptional control of CSU52. The antibody optionally can have either an antagonistic or agonistic function as compared to the antigen. This invention also contemplates antibodies that compete for binding to natural CSU53 interactors. For example, an antibody of the present invention can compete with CSU53 for binding to another protein or biological molecule, such as a nucleic acid that is under the transcriptional control of CSU53. This invention also contemplates antibodies that compete for binding to natural CSU55 interactors. For example, an antibody of the present invention can compete with CSU55 for binding to another protein or biological molecule, such as a nucleic acid that is under the transcriptional control of CSU55. The antibodies of the present invention optionally can have either an antagonistic or agonistic function as compared to the antigen.

Preferably, the antibody binds a CSU polypeptide of the present invention in vitro, ex vivo or in vivo. Optionally, the antibody of the invention is labeled with a detectable moiety. For example, the detectable moiety can be selected from the group consisting of a fluorescent moiety, an enzyme-linked moiety, a biotin moiety and a radiolabeled moiety. The antibody can be used in techniques or procedures such as diagnostics or screening. Anti-idiotypic antibodies and affinity matured antibodies are also considered to be part of the invention.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse or other species. The heavy chain constant domains that correspond to the different classes of immunoglobulins are called alpha, delta, epsilon, gamma, and mu, respectively.

As used herein, the terms “immunoglobulin heavy chain or fragments thereof” and “immunoglobulin light chain or fragments thereof” encompass chimeric peptides and hybrid peptides, with dual or multiple antigen or epitope specificities, and fragments, including hybrid fragments. Thus, fragments of the heavy chains and/or fragments of the light chains that retain the ability to bind their specific antigens are provided. For example, fragments of the heavy chains and/or fragments of the light chains that maintain CSU51 protein binding activity are included within the meaning of the terms “immunoglobulin heavy chain or fragments thereof” and “immunoglobulin light chain and fragments thereof,” respectively. Such heavy chains and light chains and fragments thereof, respectively, can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain CSU protein binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. In one embodiment of the invention, the “humanized” antibody is a human version of the antibody produced by a germ line mutant animal. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, the present invention provides a humanized version of an antibody, comprising at least one, two, three, four, or up to all CDRs of a CSU51 monoclonal antibody. In another embodiment, the present invention provides a humanized version of an antibody, comprising at least one, two, three, four, or up to all CDRs of a CSU52 monoclonal antibody. Also provided is a humanized version of an antibody, comprising at least one, two, three, four, or up to all CDRs of a CSU53 monoclonal antibody. Further provided is a humanized version of an antibody, comprising at least one, two, three, four, or up to all CDRs of a CSU55 monoclonal antibody. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

The present invention further provides a hybridoma cell that produces a monoclonal antibody of the invention. An example of such a hybridoma cell is a hybridoma cell which produces a monoclonal antibody that specifically binds an epitope contained within amino acids 1-84 of the CSU51 polypeptide (SEQ ID NO: 1). Other examples include a hybridoma cell which produces a monoclonal antibody that specifically binds an epitope contained within the amino acid sequence of a CSU52 polypeptide (SEQ ID NO: 6), a CSU 53 polypeptide (SEQ ID NO: 7) or a CSU55 polypeptide (SEQ ID NO: 8).

The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies of the invention may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises a polypeptide of the present invention.

Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of a CSU protein, for example, expressed as a fusion protein with human IgG1 is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick I E et al. High-affinity monoclonal antibodies to PED/PEA-15 generated using 5 microg of DNA. Hybridoma. 2000 August; 19(4):297-302, which are incorporated herein by reference in full for the methods of antibody production).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of a monoclonal antibody or fragment thereof of the invention and one or more reagents for detecting binding of the antibody or fragment thereof to a CSU polypeptide. The reagents can include, for example, fluorescent tags, enzymatic tags, or other tags. The reagents can also include secondary or tertiary antibodies or reagents for enzymatic reactions, wherein the enzymatic reactions produce a product that can be visualized.

### Detection Methods

The present invention provides a method for detecting a Candida albicans nucleic acid in a sample, comprising analyzing a sample for the presence of a CSU nucleic acid or expression of a CSU nucleic acid. As utilized herein, “expression” refers to the transcription of a CSU gene to yield a CSU nucleic acid, such as CSU51 mRNA, CSU52, CSU53 mRNA or CSU55 mRNA. The term “expression” also refers to the transcription and translation of a gene to yield the encoded protein, in particular a CSU51 polypeptide, CSU52 polypeptide, CSU53 polypeptide, CSU55 polypeptide or a fragment of any of these polypeptides. Therefore, one of skill in the art can detect the expression of CSU51 by monitoring CSU51 nucleic acid production and/or expression of the CSU51 protein. One of skill in the art can also detect expression of CSU52, CSU53 or CSU55 by monitoring nucleic acid production and/or expression of CSU52, CSU53 or CSU55, respectively. One of skill in the art can also determine if a CSU nucleic acid or gene is present in a sample by probing or amplifying genomic DNA in the sample.

The amount of a nucleic acid encoding a CSU polypeptide in a cell can be determined by methods standard in the art for detecting or quantitating nucleic acid in a cell, such as in situ hybridization, quantitative PCR, Southern blotting, Northern blotting, ELISPOT, dot blotting, etc., as well as any other method now known or later developed for detecting or quantitating the amount of a nucleic acid in a cell.

The presence or amount of CSU protein (for example, CSU51, CSU52, CSU53 or CSU55), in a cell can be determined by methods standard in the art, such as Western blotting, ELISA, ELISPOT, immunoprecipitation, immunofluorescence (e.g., FACS), immunohistochemistry, immunocytochemistry, etc., as well as any other method now known or later developed for detecting or quantitating protein in or produced by a cell.

The sample of this invention can be a sample from a cell culture, for example a yeast cell culture from any yeast species, such as a C. albicans culture, or from any organism and can be, but is not limited to, peripheral blood, urine, vaginal secretions, a pap smear sample, bone marrow specimens, primary tumors, embedded tissue sections, frozen tissue sections, cell preparations, cytological preparations, exfoliate samples (e.g., sputum), fine needle aspirations, lung fluid, amnion cells, fresh tissue, dry tissue, and cultured cells or tissue. The sample can be from or near the site of a fungal infection, such as, but not limited to a Candida albicans infection, in an organism. The sample can be from malignant tissue or non-malignant tissue. The sample can be unfixed or fixed according to standard protocols widely available in the art and can also be embedded in a suitable medium for preparation of the sample. For example, the sample can be embedded in paraffin or other suitable medium (e.g., epoxy or acrylamide) to facilitate preparation of the biological specimen for the detection methods of this invention. Furthermore, the sample can be embedded in any commercially available mounting medium, either aqueous or organic.

The sample can be on, supported by, or attached to, a substrate which facilitates detection. A substrate of the present invention can be, but is not limited to, a microscope slide, a culture dish, a culture flask, a culture plate, a culture chamber, ELISA plates, as well as any other substrate that can be used for containing or supporting biological samples for analysis according to the methods of the present invention. The substrate can be of any material suitable for the purposes of this invention, such as, for example, glass, plastic, polystyrene, mica and the like. The substrates of the present invention can be obtained from commercial sources or prepared according to standard procedures well known in the art.

Conversely, an antibody or fragment thereof, an antigenic fragment of a CSU polypeptide, or a CSU nucleic acid of the invention can be on, supported by, or attached to a substrate which facilitates detection. Such a substrate can include a chip, a microarray or a mobile solid support. Thus, provided by the invention are substrates including one or more of the antibodies or antibody fragments, antigenic fragments of CSU polypeptides, or CSU nucleic acids of the invention.

For example, the present invention provides a method of detecting the presence of Candida albicans in a sample comprising: a) contacting a sample with an antibody to a CSU51 polypeptide; and b) detecting the antibody bound to the CSU51 polypeptide in the sample, wherein binding of CSU51 polypeptide to the antibody indicates the presence of Candida albicans in the sample.

The present invention also provides a method of detecting the presence of Candida albicans in a sample comprising: a) contacting a sample with an antibody to a CSU52 polypeptide; and b) detecting the antibody bound to the CSU52 polypeptide in the sample, wherein binding of CSU52 polypeptide to the antibody indicates the presence of Candida albicans in the sample.

Further provided is a method of detecting the presence of Candida albicans in a sample comprising: a) contacting a sample with an antibody to a CSU53 polypeptide; and b) detecting the antibody bound to the CSU53 polypeptide in the sample, wherein binding of CSU53 polypeptide to the antibody indicates the presence of Candida albicans in the sample.

For example, the present invention provides a method of detecting the presence of Candida albicans in a sample comprising: a) contacting a sample with an antibody to a CSU55 polypeptide; and b) detecting the antibody bound to the CSU55 polypeptide in the sample, wherein binding of CSU55 polypeptide to the antibody indicates the presence of Candida albicans in the sample.

These methods can be used to detect the presence of other Candida species in a sample by contacting a sample with an antibody to a homolog of a CSU polypeptide described herein and detecting the antibody bound to the CSU homolog in the sample. For example, this method can be utilized to detect Candida tropicalis, Candida glabrata, Candida parapsilosis, Candida krusei, Candida lusitaniae, Candida kefyr, Candida guilliermondii, Candida dubliniensis, Candida ciferri, Candida famata, Candida lambica, Candida lipolytica, Candida norvegensis, Candida rugosa, Candida viswanathii and Candida zeylanoide.

The nucleic acids of this invention can be detected with a probe capable of hybridizing to the nucleic acid of a cell or a sample. This probe can be a nucleic acid comprising the nucleotide sequence of a coding strand or its complementary strand or the nucleotide sequence of a sense strand or antisense strand, or a fragment thereof. The nucleic acid can comprise the nucleic acid of a CSU51 gene, the nucleic acid of a CSU52 gene, the nucleic acid of a CSU53 gene, the nucleic acid of a CSU55 gene or a fragment of one of these nucleic acids. Thus, the probe of this invention can be either DNA or RNA and can bind either DNA or RNA, or both, in the biological sample. The probe can be the coding or complementary strand of a complete CSU gene or CSU gene fragment.

Therefore, the present invention provides a method for detecting the presence of Candida albicans in a sample, comprising the steps of: (a) contacting a biological sample with a probe under conditions that allow the probe to selectively bind a CSU51 nucleic acid; and (b) detecting the presence of a CSU51 nucleic acid, whereby the presence a CSU51 nucleic acid indicates the presence of Candida albicans in the sample. The present invention also provides a method for detecting the presence of Candida albicans in a sample, comprising the steps of: (a) contacting a biological sample with a probe under conditions that allow the probe to selectively bind a CSU52 nucleic acid; and (b) detecting the presence of a CSU52 nucleic acid, whereby the presence a CSU52 nucleic acid indicates the presence of Candida albicans in the sample. Also provided is a method for detecting the presence of Candida albicans in a sample, comprising the steps of: (a) contacting a biological sample with a probe under conditions that allow the probe to selectively bind a CSU53 nucleic acid; and (b) detecting the presence of a CSU53 nucleic acid, whereby the presence a CSU53 nucleic acid indicates the presence of Candida albicans in the sample. Further provided is a method for detecting the presence of Candida albicans in a sample, comprising the steps of: (a) contacting a biological sample with a probe under conditions that allow the probe to selectively bind a CSU55 nucleic acid; and (b) detecting the presence of a CSU55 nucleic acid, whereby the presence a CSU55 nucleic acid indicates the presence of Candida albicans in the sample. These methods can be used to detect the presence of other Candida species in a sample such as Candida tropicalis, Candida glabrata, Candida parapsilosis, Candida krusei, Candida lusitaniae, Candida kefyr, Candida guilliermondii, Candida dubliniensis, Candida ciferri, Candida famata, Candida lambica, Candida lipolytica, Candida norvegensis, Candida rugosa, Candida viswanathii and Candida zeylanoide.

The nucleic acids of the present invention, for example, SEQ ID NO: 2, SEQ ID NO: 3, SEQ ID NO: 9, SEQ ID NO: 10, SEQ ID NO: 11 and fragments thereof, can be utilized as probes or primers to detect CSU nucleic acids. For example, a polynucleotide probe or primer comprising a polynucleotide of at least 15 contiguous nucleotides of SEQ ID NO: 2 can be utilized to detect a CSU51 nucleic acid. In another example, a polynucleotide probe or primer comprising a polynucleotide of at least 15 contiguous nucleotides of SEQ ID NO: 9 can be utilized to detect a CSU52 nucleic acid. Similarly, a polynucleotide probe or primer comprising a polynucleotide of at least 15 contiguous nucleotides of SEQ ID NO: 10 can be utilized to detect a CSU53 nucleic acid. Also, a polynucleotide probe or primer comprising a polynucleotide of at least 15 contiguous nucleotides of SEQ ID NO: 11 can be utilized to detect a CSU55 nucleic acid Therefore, the polynucleotide probes or primers of this invention can be at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or at least 200 nucleotides in length.

As used herein, the term “nucleic acid probe” refers to a nucleic acid fragment that selectively hybridizes under stringent conditions with a nucleic acid comprising a nucleic acid set forth in a sequence listed herein. This hybridization must be specific. The degree of complementarity between the hybridizing nucleic acid and the sequence to which it hybridizes should be at least enough to exclude hybridization with a nucleic acid encoding an unrelated protein.

Stringent conditions refers to the washing conditions used in a hybridization protocol. In general, the washing conditions should be a combination of temperature and salt concentration chosen so that the denaturation temperature is approximately 5-20° C. below the calculated Tₘ of the nucleic acid hybrid under study. The temperature and salt conditions are readily determined empirically in preliminary experiments in which samples of reference DNA immobilized on filters are hybridized to the probe or protein coding nucleic acid of interest and then washed under conditions of different stringencies. The Tₘ of such an oligonucleotide can be estimated by allowing 2° C. for each A or T nucleotide, and 4° C. for each G or C. For example, an 18 nucleotide probe of 50% G+C would, therefore, have an approximate Tₘ of 54° C.

Stringent conditions are known to one of skill in the art. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share 90% Identity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Detects Sequences that Share 80% Identity or Greater)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share Greater than 50% Identity)

| | |
|---|---|
| Hybridization: | 6x SSC at RT to 55° C. for 16-20 hours |
| Wash at least twice: | 2x-3x SSC at RT to 55° C. for 20-30 minutes each. |

Therefore, the probes derived from SEQ ID NO: 2 and SEQ ID NO: 3 can be utilized to identify other CSU51 nucleic acids in other yeast species, such as in other Candida species, by varying stringency conditions. These probes can also be utilized to screen cDNA or genomic libraries from other yeast species to identify nucleic acids that encode homologs of CSU51. Similarly, probes derived from SEQ ID NO: 9 can be utilized to identify other CSU52 nucleic acids in other yeast species, probes derived from SEQ ID NO: 10 can be utilized to identify other CSU53 nucleic acids in other yeast species and probes derived from SEQ ID NO: 11 can be utilized to identify other CSU55 nucleic acids in other yeast species.

As mentioned above, CSU nucleic acids and fragments thereof can be utilized as primers to amplify a CSU nucleic acid, such as a CSU51 gene transcript, a CSU52 transcript, a CSU53 transcript, or a CSU55 transcript by standard amplification techniques. For example, expression of any CSU gene transcript for the genes set forth herein can be quantified by RT-PCR using RNA isolated from cells.

Therefore, the present invention provides a method for detecting the presence of Candida albicans in a sample, comprising the steps of: (a) contacting a biological sample with at least two oligonucleotide primers, each primer consisting of 10 to 200 contiguous nucleotides of SEQ ID NO: 2, SEQ ID NO: 3 or the complement thereof, in a reverse transcriptase polymerase chain reaction; and (b) detecting in the sample a polynucleotide sequence that amplifies in the presence of said oligonucleotide primers, wherein the presence of an amplified polynucleotide sequence indicates the presence of Candida albicans in the sample. Examples of primers that can be utilized to amplify a CSU51 nucleic acid sequence include but are not limited to forward primer (5′-CAA TTC ACC AAA GTT ATC GCT-3′) (SEQ ID NO: 4) and reverse primer (5′-GTA CAA TAA AGC AGC ACC AAC-3′) (SEQ ID NO: 5).

Also provided is a method for detecting the presence of Candida albicans in a sample, comprising the steps of: (a) contacting a biological sample with at least two oligonucleotide primers, each primer consisting of 10 to 200 contiguous nucleotides of SEQ ID NO: 9 or the complement thereof, in a reverse transcriptase polymerase chain reaction; and (b) detecting in the sample a polynucleotide sequence that amplifies in the presence of said oligonucleotide primers, wherein the presence of an amplified polynucleotide sequence indicates the presence of Candida albicans in the sample.

Also provided is a method for detecting the presence of Candida albicans in a sample, comprising the steps of: (a) contacting a biological sample with at least two oligonucleotide primers, each primer consisting of 10 to 200 contiguous nucleotides of SEQ ID NO: 10 or the complement thereof, in a reverse transcriptase polymerase chain reaction; and (b) detecting in the sample a polynucleotide sequence that amplifies in the presence of said oligonucleotide primers, wherein the presence of an amplified polynucleotide sequence indicates the presence of Candida albicans in the sample.

Also provided is a method for detecting the presence of Candida albicans in a sample, comprising the steps of: (a) contacting a biological sample with at least two oligonucleotide primers, each primer consisting of 10 to 200 contiguous nucleotides of SEQ ID NO: 11 or the complement thereof, in a reverse transcriptase polymerase chain reaction; and (b) detecting in the sample a polynucleotide sequence that amplifies in the presence of said oligonucleotide primers, wherein the presence of an amplified polynucleotide sequence indicates the presence of Candida albicans in the sample.

A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods. One of skill in the art would know how to design and synthesize primers that amplify the CSU nucleic acids of the present invention based on the nucleic acids sequences encoding CSU51 (SEQ ID NO: 2), CSU52 (SEQ ID NO: 9), CSU53 (SEQ ID NO: 10) and CSU55 (SEQ ID NO: 11).

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified fragment, can be analyzed by one of a number of methods known in the art. The nucleic acid can be sequenced by dideoxy or other methods. Hybridization with the sequence can also be used to determine its presence, by Southern blots, dot blots, etc.

The CSU nucleic acids of the present invention can also be used in polynucleotide arrays. Polynucleotide arrays provide a high throughput technique that can assay a large number of polynucleotide sequences in a single sample. This technology can be used, for example, as a diagnostic tool to identify samples that contain a CSU nucleic acid as well as samples with increased or decreased expression of a CSU gene transcript or CSU polypeptide.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from the nucleotide sequences shown in SEQ ID NOS: 2, 3, 9, 10 and 11.

The substrate can be any substrate to which polynucleotide probes can be attached, including but not limited to glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Commercially available polynucleotide arrays, such as Affymetrix GeneChip™, can also be used. Use of the GeneChip™ to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

Tissue samples can be treated to form single-stranded polynucleotides, for example by heating or by chemical denaturation, as is known in the art. The single-stranded polynucleotides in the tissue sample can then be labeled and hybridized to the polynucleotide probes on the array. Detectable labels which can be used include but are not limited to radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance.

Since therapy and clinical decisions are often dependent on diagnosis, detection of CSU expression utilizing the methods described herein, allows one of skill in the art to detect the presence of a CSU gene or CSU expression in a sample from a subject. For example, and not to be limiting, since CSU51 is not found in humans, the presence of CSU51 indicates the presence of a Candida infection. The presence of a CSU51 gene and/or CSU51 expression in a sample indicates the presence of Candida albicans in the subject. One skilled in the art would be able to measure CSU51 levels in numerous subjects in order to establish ranges of CSU51 levels that correspond to stages of Candida albicans infection. One of skill in the art can then determine which therapy to administer depending on the levels of CU51 present in the sample that correlate with a particular stage of infection. This can also be done for CSU52, CSU53 and CSU55. One or more of the CSU nucleic acids or polypeptides set forth herein can be detected to determine the presence of Candida albicans. Any of the detection methods described herein can be used in combination with other detection methods now know or established in the future to confirm the presence or absence of a Candida albicans infection.

### Modified Candida Cells

The present invention provides a Candida albicans cell comprising a mutated CSU nucleic acid sequence. Also provided are cells from other Candida species comprising a mutated CSU nucleic acid sequence. For example, the gene encoding CSU51, CSU52, CSU53 or CSU55 can comprise one or more point mutations, a deletion mutation or an insertion. One or more CSU genes can be mutated in these cells. One or more genes encoding CSU51, CSU52, CSU53 or CSU55 can also be knocked out. One or two copies of a nucleic acid or gene encoding a CSU polypeptide can be mutated, deleted or knocked out to provide a Candida albicans cell comprising a mutated CSU nucleic acid sequence. Since CSU51 is part of a redundant regulatory pathway, the present invention also provides cells comprising a mutated CSU51 nucleic acid sequence and a mutation in one or more CSU genes in regions of chromosome 5 selected from the group consisting of: region A of sequence 140, region B of Sequence 140, sequence 135 and region C. These regions can comprise a point mutation, a deletion mutation, or an insertion. One of more copies of each of these regions or CSU genes contained within these regions can be mutated, deleted or knocked out to provide a Candida cell comprising a mutated CSU51 nucleic acid sequence and an additional mutation in one or more of region A of sequence 140, region B of Sequence 140, sequence 135 and region C. It is noted that although the present invention shows that deletion of CSU51 in combination with other regions of chromosome 5 (as described in the Examples) was required in order to obtain growth on sorbose, the invention is not limited to requiring CSU51 to be targeted, deleted, mutated etc. with another CSU gene on chromosome 5. Thus the invention is directed to the effects of CSU51 alone or in combination with other CSU genes and is not limited to the effects seen in a sorbose utilization model. Similarly, the present invention is also directed to the effects of CSU52, CSU53 or CSU55, alone or in combination with other CSU genes and is not limited to the effects seen in a sorbose utilization model.

The present invention also provides methods of reducing expression of CSU51, CSU52, CSU53 or CSU55 in cells, including Candida albicans cells, and the cells produced by these methods. A method of reducing expression of one or more regulators selected from the group consisting of CSU51, CSU52, CSU53 and CSU55 in cells is also provided by the present invention. Also provided are cells where CSU51 expression is reduced and expression of one or more CSU negative regulators from region A of sequence 140, region B of Sequence 140, sequence 135 or region C of chromosome 5 is also reduced. These cells can be utilized to determine the effects of reducing CSU51 expression (either alone or in combination with reduction of expression of other CSU genes) on any activity of CSU51, yeast cell growth, yeast cell fitness (for example, determining if the cell is weakened upon reduction of CSU51 expression) morphology, nutrient utilization, pathogenesis, genes under the transcriptional control of CSU51, for example SOU1, (either directly by binding of CSU51 to promoters or indirectly by binding of CSU51 to other transcriptional factors that control expression of genes) and any other property of a Candida albicans cell or a Candida albicans gene. These cells can also be used to determine the effects of reducing CSU51 expression (either alone or in combination with the reduction of expression of other CSU genes) on the ability of a Candida albicans cell to grow in the presence of other natural microflora (for example, the natural microflora found in the mouth gut and intestine (E. coli)) and pathogens (for example, yeasts, bacteria, and viruses). In a nonlimiting example, reduction of expression of CSU51, CSU52, CSU53 or CSU55, either alone or in combination with reduction of expression of another CSU gene on chromosome 5 can result in the inability of a yeast cell to grow in the presence of naturally occurring microflora such as E. coli. If so, the yeast cell now has a reduced ability to compete for nutrients with healthy microflora and is eliminated from the organism. It is noted that the effects of mutating a CSU51 nucleic acid and/or other CSU genes on cell growth are not limited to growth on sorbose and include growth on agar, dextrose, arabinose etc.

The present invention also provides a Candida albicans cell wherein a CSU polypeptide is overexpressed. Also provided are cells from other Candida species that overexpress a CSU polypeptide. These cells can be utilized to determine the effects of increasing CSU51 expression (either alone or in combination with increasing the expression of other CSU genes described above) on any activity of CSU51, yeast cell growth, yeast cell fitness (for example, determining if the cell is weakened upon increasing CSU51 expression) morphology, nutrient utilization, pathogenesis, genes under the transcriptional control of CSU51, for example SOU1, (either directly by binding of CSU51 to promoters or indirectly by binding of CSU51 to other transcriptional factors that control expression of genes) and any other property of a Candida albicans cell or a Candida albicans gene. These cells can also be utilized to determine the effects of increasing expression of CSU52, CSU53 or CSU55, as described above for CSU51. These cells can also be used to determine the effects of increasing CSU51 expression (either alone or in combination with increased expression of other CSU genes) on the ability of a Candida albicans cell to grow in the presence of other natural microflora (for example, the natural microflora found in the mouth gut and intestine (E. coli)) and pathogens (for example, yeasts, bacteria, and viruses). Similarly, these cells can also be used to determine the effects of increasing expression of CSU52, CSU53 or CSU55 on the ability of a Candida albicans cell to grow in the presence of other natural microflora, as described above for CSU51.

The present invention provides a method of identifying an agent that modulates an activity of CSU polypeptide comprising the steps of: a) contacting a host cell comprising a polynucleotide sequence that encodes a functional CSU polypeptide with a test agent; and b) determining if at least one activity of the CSU polypeptide is modulated, such that if an activity of the CSU polypeptide is modulated, the test agent is an agent that modulates CSU polypeptide activity. The CSU polypeptides that can be utilized in these methods include, but are not limited to CSU51, CSU52, CSU53 and CSU55. As utilized in these methods, host cells are not limited to yeast cells, as other eukaryotic cells engineered to express a CSU polypeptide can also be used to screen for agents that modulate expression and/or activity of a CSU polypeptide. In the modulation of an activity of a CSU polypeptide, an activity can be increased or decreased. For example, an agent can increase or decrease expression of a CSU polypeptide. In another example, an agent can decrease expression of a CSU polypeptide, for example CSU51, resulting in increased expression of other genes (for example, SOU1) that are negatively regulated by the CSU polypeptide and decreased expression of other genes that are positively regulated by the CSU polypeptide. In another example, an agent can increase the expression of a CSU polypeptide, for example, CSU51, resulting in decreased expression of genes (for example, SOU1) negatively regulated by the CSU polypeptide and increased expression of genes that are positively regulated by the CSU polypeptide.

As utilized herein, “activity of a CSU polypeptide” refers to an activity or characteristic associated with expression of a CSU polypeptide. For the CSU polypeptides set forth herein, this activity can be termed “CSU51 activity,” “CSU52 activity,” “CSU53 activity,” or “CSU55 activity.” The nature of this activity(s) or characteristic(s) can depend on transcriptional regulation (i.e. repression or negative regulation) of certain genes. These activities and characteristics include, but are not limited to, expression of a CSU polypeptide (i.e., transcription and translation of a CSU polypeptide, for example, CSU51, CSU52, CSU53 or CSU55), binding other proteins (particularly DNA binding proteins), regulation (whether induction or repression) of certain genes, and particular phenotypic characteristics. Because a CSU polypeptide of the present invention exerts control over a number of other genes, it is understood that the term “CSU activity” encompasses results and characteristics that stem from expression of a CSU polypeptide which include affecting gene expression of any gene(s) that is regulated by a gene product of a CSU polypeptide or an active fragment thereof. For example, since SOU1 is repressed by expression of CSU51, then lack of expression of SOU1 is an activity of CSU51.

As used herein, a characteristic which is associated with a “loss or decrease in activity” is a characteristic which is associated with a decrease in the function of a CSU gene or polypeptide. This decrease may range from partial to total loss, or knockout, of function. A decrease in activity can occur as a result of an effect at any point along any pathway in which a CSU polypeptide exerts control, from transcription of the CSU gene, to expression (i.e., transcription and/or translation), to affecting regulation of any gene(s) under control of the CSU polypeptide, to activity associated with regulation of these gene(s).

The test agents of the present invention include but are not limited to, a chemical, a drug, a small molecule, a small polypeptide, a large polypeptide, an antisense oligonucleotide, a ribozyme, a siRNA, an antibody, cDNAs, compound libraries and the like. The screening methods described herein can be automated and optimized for high throughput screening of test agents. Any of the agents identified by the methods of the present invention as modulators of CSU51, CSU52, CSU53 or CSU55 can be utilized as an antifungal agent to treat a Candida infection. Such an infection can be localized or it can be a systemic infection. Also, any of the agents identified by the methods of the present invention can also be screened for their ability to treat a fluconazole resistant fungal infection. Any of the agents identified by the methods described herein can be administered to a fluconazole resistant strain or any other strain that exhibits drug resistance, to determine its effects. If the agent reduces cell growth, decreases pathogenicity or otherwise affects these strains such that their growth or infective properties are decreased, the agent can be used as a therapeutic agent to treat a drug resistant Candida strain. Furthermore, any of the CSU51 modulators identified by the methods of the present invention can be utilized in combination with other CSU gene modulators, for example, modulators of CSU52-55. The CSU51 modulators, CSU52 modulators, CSU53 modulators and CSU55 modulators of the present invention can also be used in combination with other antifungal agents such as flucanazole.

### Identification of Genes in the CSU Pathway

The invention also provides methods for cloning genes and gene products that are involved in, and/or associated with, a CSU51 activity (i.e., a CSU51 functional pathway). Because CSU51 has been shown to play an important role in sorbose utilization and its sequence is not found in humans, genes that are involved in a CSU51 pathway may well be suitable and useful drug targets. Further, these gene(s) and gene product(s) may provide even more precise, specific targets for drug discovery and development, and hence therapy. The polynucleotides encoding these genes may also be less conserved among fungi and even among various species of Candida, and thus may be especially suitable diagnostic reagents.

Accordingly, the invention provides methods of isolating genes involved in a CSU51 pathway comprising: (a) identification of nucleic acid sequences which are repressed or activated upon CSU51 expression in Candida albicans. For these methods, the polynucleotides are identified using standard techniques in the art for determining differential expression. These methods can optionally include an additional step of (b) identifying those sequences from step (a) above which are expressed when C. albicans is grown on sorbose. Presumably, this sequence is then considered to be required for sorbose utilization and thus associated with CSU51 activity. Similar techniques can be utilized to identify sequences that are required for sorbose utilization and associated with CSU52 activity, CSU53 activity or CSU55 activity.

As described above, the present invention provides one or more CSU genes, including CSU51, CSU52, CSU53 and CSU55 that are involved in redundant negative regulatory pathways. Therefore, the present invention provides for the identification of other CSU genes on chromosome 5, specifically in region A of sequence 140, region B of Sequence 140, sequence 135 and region C that are involved in the CSU51 pathway, in addition to CSU51, CSU52, CSU53 and CSU55. Therefore, the present invention also provides for identification of nucleic acid sequences which are repressed or activated upon CSU51 expression, either alone or in combination with expression of other CSU genes in Candida albicans. Similarly, the present invention also provides for identification of nucleic acid sequences which are repressed or activated upon CSU52 expression, CSU53 expression, or CSU55 expression, either alone or in combination with expression of other CSU genes in Candida albicans. For these methods, the polynucleotides are identified using standard techniques in the art for determining differential expression.

### Methods of Reducing Cell Growth

The present invention provides a method of inhibiting Candida albicans cell growth comprising administering to the cell an agent that reduces expression of a CSU polypeptide. All of the methods described herein can also be utilized to decrease expression of one or more CSU polypeptides selected from the group consisting of CSU51, CSU52, CSU53 and CSU55. As utilized herein, “suppression or inhibition of cell growth” means a decrease in cell growth which can be, but is not limited to a decrease in cell division, a decrease in the number of cells and/or a decrease in cell size as compared to a control. The control can be a control cell that is not contacted with the agent. This decrease does not have to be complete and can range from a slight decrease in cell growth to a complete elimination or arrest of cell growth. The cell can be in vitro, ex vivo or an in vivo cell. The cell can be a cell engineered to express a CSU polypeptide (for example, CSU51, CSU52, CSU53 or CSU55) or a functional fragment thereof. The cell can also be a cell that normally expresses a CSU polypeptide or a functional fragment thereof, such as a Candida albicans cell.

Therefore, the present invention provides a method of reducing expression of a CSU polypeptide in a cell comprising administering to the cell an antisense oligonucleotide that specifically binds to mRNA transcribed from the CSU gene under conditions that allow hybridization. For example, provided herein is a method of reducing expression of CSU51 in a cell comprising administering to the cell an antisense oligonucleotide that specifically binds to mRNA transcribed from the CSU51 gene under conditions that allow hybridization, wherein the CSU51 mRNA comprises SEQ ID NO: 2 or an mRNA that is at least 50% identical to SEQ ID NO: 2. Also provided is a method of reducing expression of CSU52 in a cell comprising administering to the cell an antisense oligonucleotide that specifically binds to mRNA transcribed from the CSU52 gene under conditions that allow hybridization, wherein the CSU52 mRNA comprises SEQ ID NO: 9 or an mRNA that is at least 50% identical to SEQ ID NO: 9. Further provided is a method of reducing expression of CSU53 in a cell comprising administering to the cell an antisense oligonucleotide that specifically binds to mRNA transcribed from the CSU53 gene under conditions that allow hybridization, wherein the CSU53 mRNA comprises SEQ ID NO: 10 or an mRNA that is at least 50% identical to SEQ ID NO: 10. Also provided is a method of reducing expression of CSU55 in a cell comprising administering to the cell an antisense oligonucleotide that specifically binds to mRNA transcribed from the CSU55 gene under conditions that allow hybridization, wherein the CSU55 mRNA comprises SEQ ID NO:11 or an mRNA that is at least 50% identical to SEQ ID NO: 11.

As used herein, “reducing” or “inhibiting” means a decrease in expression levels. Such reduction does not have to be complete and can range from a slight decrease in expression to complete inhibition of expression.

Antisense technology is well known in the art and describes a mechanism whereby a nucleic acid comprising a nucleotide sequence which is in a complementary, “antisense” orientation with respect to a coding or “sense” sequence of an endogenous gene, is introduced into a cell, whereby a duplex may form between the antisense sequence and its complementary sense sequence. The formation of this duplex may result in inactivation of the endogenous gene.

For example, the antisense nucleic acid can inhibit gene expression by forming an RNA/RNA duplex between the antisense RNA and the RNA transcribed from a target gene. The precise mechanism by which this duplex formation decreases the production of the protein encoded by the endogenous gene most likely involves binding of complementary regions of the normal sense mRNA and the antisense RNA strand with duplex formation in a manner that blocks RNA processing and translation. Alternative mechanisms include the formation of a triplex between the antisense RNA and duplex DNA or the formation of a DNA-RNA duplex with subsequent degradation of DNA-RNA hybrids by RNAse H. Furthermore, an antisense effect can result from certain DNA-based oligonucleotides via triple-helix formation between the oligomer and double-stranded DNA which results in the repression of gene transcription. Antisense nucleic acid can be produced for any relevant endogenous gene for which the coding sequence has been or can be determined according to well known methods.

A nucleic acid encoding an antisense RNA can be selected based on the protein desired to be inhibited or decreased in cells, by providing an RNA that will selectively bind to the cellular mRNA encoding such protein. Binding of the antisense molecule to the target mRNA may incapacitate the mRNAs, thus preventing its translation into a functional protein. The antisense RNA/mRNA complexes can then become a target for RNAse-H and are eventually degraded by the host cell RNAse-H. Control regions, such as enhancers and promoters, can be selected for antisense RNA targeting according to the cell or tissue in which it is to be expressed, as is known in the art. Preferable antisense-encoding constructs can encode full-length complements to target sequences; however, smaller length sequences down to oligonucleotide size can be utilized. For example, the antisense-encoding constructs can encode full-length complements to the CSU51 gene, smaller length sequences or oligonucleotide sequences.

Also provided is a method of reducing expression of CSU polypeptide, such as CSU51, CSU52, CSU53 or CSU55, comprising administering to a cell a ribozyme that specifically binds to mRNA transcribed from a CSU gene, the ribozyme binding reducing expression of the CSU polypeptide.

Therefore, expression of CSU51, CSU52, CSU53 or CSU55 can be decreased using a ribozyme, an RNA molecule with catalytic activity. See, e.g., Cech, 1987, Science 236: 1532-1539; Cech, 1990, Ann. Rev. Biochem. 59:543-568; Cech, 1992, Curr. Opin. Struct. Biol. 2: 605-609; Couture and Stinchcomb, 1996, Trends Genet. 12: 510-515. Ribozymes can be used to inhibit gene function by cleaving an RNA sequence, as is known in the art (e.g., Haseloff et al., U.S. Pat. No. 5,641,673).

The coding sequence of the CSU polypeptides set forth herein can be used to generate a ribozyme which will specifically bind to mRNA transcribed from a CSU gene. Methods of designing and constructing ribozymes which can cleave other RNA molecules in trans in a highly sequence specific manner have been developed and described in the art (see Haseloff et al. (1988), Nature 334:585-591). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201). Longer complementary sequences can be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target.

Ribozymes can be introduced into cells as part of a DNA construct, as is known in the art. The DNA construct can also include transcriptional regulatory elements, such as a promoter element, an enhancer or UAS element, and a transcriptional terminator signal, for controlling the transcription of the ribozyme in the cells.

Mechanical methods, such as microinjection, liposome-mediated transfection, electroporation, or calcium phosphate precipitation, can be used to introduce the ribozyme-containing DNA construct into cells whose division it is desired to decrease, as described above. Alternatively, if it is desired that the DNA construct be stably retained by the cells, the DNA construct can be supplied on a plasmid and maintained as a separate element or integrated into the genome of the cells, as is known in the art.

As taught in Haseloff et al., U.S. Pat. No. 5,641,673, the ribozyme can be engineered so that its expression will occur in response to factors which induce expression of CSU51. The ribozyme can also be engineered to provide an additional level of regulation, so that destruction of mRNA occurs only when both the ribozyme and the CSU51 gene are induced in the cells.

Further provided is a method of reducing CSU polypeptide expression comprising administering to a cell an siRNA that is complementary to at least a portion of the coding sequence of a CSU polypeptide described herein, under conditions that allow hybridization of the siRNA with the CSU polypeptide coding sequence, wherein binding of the siRNA to the CSU polypeptide coding sequence reduces CSU polypeptide expression. For example, an siRNA that is complementary to the CSU51 coding sequence set forth as SEQ ID NO: 2 can be utilized in these methods. In another example, an siRNA that is complementary to the CSU52 coding sequence set forth as SEQ ID NO: 9 can be utilized. In yet another example, an siRNA that is complementary to the CSU53 coding sequence set forth as SEQ ID NO: 10 can be utilized. Furthermore, an siRNA that is complementary to the CSU55 coding sequence set forth as SEQ ID NO: 11 can be utilized.

Methods of inhibiting or reducing CSU polypeptide expression as well as methods of inhibiting or reducing CSU polypeptide activity can be utilized to treat subjects with a Candida infection, such as a Candida albicans infection. As used herein, “treating” describes an improvement in the patient's clinical state. The improvement may range from reduction of the symptoms of the disease to complete amelioration of the disease.

Various delivery systems for administering the therapies disclosed herein are known, and include encapsulation in liposomes, microparticles, microcapsules, expression by recombinant cells, receptor-mediated endocytosis (Wu and Wu, J. Biol. Chem. 1987, 262:4429-32), and construction of therapeutic nucleic acids as part of a retroviral or other vector. Methods of introduction include, but are not limited to, topical, intradermal, intramuscular, intraperitoneal, intravenous, subcutaneous, intranasal, and oral routes. The compounds can be administered by any convenient route, for example by infusion or bolus injection, by absorption through epithelial or mucocutaneous linings (for example, oral mucosa, rectal, vaginal and intestinal mucosa, etc.) and can be administered together with other biologically active agents. Administration can be systemic or local. Pharmaceutical compositions can be delivered locally to the area in need of treatment, for example by topical application.

Pharmaceutical compositions are disclosed that include a therapeutically effective amount of an RNA, DNA, antisense molecule, ribozyme, siRNA, molecule, specific-binding agent, or other therapeutic agent, alone or with a pharmaceutically acceptable carrier. Furthermore, the pharmaceutical compositions or methods of treatment can be administered in combination with (such as before, during, or following) other therapeutic treatments, such as other antifungal agents.

### Delivery Systems

The pharmaceutically acceptable carriers useful herein are conventional.

Remington's Pharmaceutical Sciences, by Martin, Mack Publishing Co., Easton, Pa., 15th Edition (1975), describes compositions and formulations suitable for pharmaceutical delivery of the therapeutic agents herein disclosed. In general, the nature of the carrier will depend on the mode of administration being employed. For instance, parenteral formulations usually include injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. For solid compositions (for example powder, pill, tablet, or capsule forms), conventional non-toxic solid carriers can include, for example, pharmaceutical grades of mannitol, lactose, starch, sodium saccharine, cellulose, magnesium carbonate, or magnesium stearate. The composition can be formulated as a suppository, with traditional binders and carriers such as triglycerides.

Embodiments of the disclosure including medicaments can be prepared with conventional pharmaceutically acceptable carriers, adjuvants and counterions as would be known to those of skill in the art.

The amount of therapeutic agent effective in decreasing or inhibiting fungal infection can depend on the nature of the fungus and its associated disorder or condition, and can be determined by standard clinical techniques. In addition, in vitro assays can be employed to identify optimal dosage ranges. The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

In an example in which a nucleic acid is employed to reduce fungal infection, such as an antisense or siRNA molecule, the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). The present disclosure includes all forms of nucleic acid delivery, including synthetic oligos, naked DNA, plasmid and viral delivery, integrated into the genome or not.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLES

A reversible decrease or increase of Candida albicans chromosome copy number was found to be a prevalent means of survival of this opportunistic pathogen under conditions that kill cells or inhibit their propagation. The utilization of a secondary carbon source, L-sorbose, by reversible loss of chromosome 5 serves as a model system. This system and negative regulators associated with the sorbose utilization pathway can be involved in pathogenesis. The present invention provides an approximately 209 kbp portion of the right arm of chromosome 5 that contains at least five spatially separated functionally redundant regions that control utilization of L-sorbose. The regions bear no structural similarity among themselves, and four of them contain sequences that bear no similarity with any known sequence. The present invention provides a regulatory gene that was found in Region A of chromosome 5, which encodes a novel helix-loop-helix protein. The multiple redundant regulators that are scattered along chromosome 5 explain in a simple elegant way, why the loss of the entire homologue is usually required for the growth on sorbose. Thus, an entire chromosome acts as a single regulatory unit, a feature not previously considered. This finding can be a paradigm for the control of other phenotypes in C. albicans that also depend on chromosome loss, thus implying that C. albicans genes are not distributed randomly among different chromosomes.

### Strains, Plasmids, Primers, Media, Growth Condition, and Software

The C. albicans Ura⁻ Sou⁻ strain CAF4-2 and prototrophic Sou strain 3153A, as well as their Ura⁻ Sou⁺ mutant Sor19 lacking one chromosome 5 and the exceptional Sou⁺ mutant Sor5, respectively, were previously reported (3, 8, 11).

All plasmids used in this work are derivatives of pSFU1, pSFI1 (12, 13), pABTEL, pAK45, pAK104 (14), and pCA88 (9). Also, pRC3915 was previously published (15). All primers are presented in Table 3.

Yeast extract/peptone/dextrose (YPD), synthetic dextrose (SD), L-sorbose media (5), as well as yeast carbon base/bovine serum albumin (YCB-BSA) rich medium and SD medium containing the mycophenolic acid (MPA) at 8 μg/ml have been previously described (13). In order to prepare solid medium, 2% (w/v) agar was added. Uridine, 50 μg/ml, was added when needed. The growth and handling of cells have been previously described (6, 9, 16).

DNA and protein sequences were analyzed with the software available on the sites of National Center for Biotechnology and Information (NCBI) (http://www.ncbi.nlm.nih.gov), Stanford Genome Technology Center (http://www-sequence.stanford.edu); NRC-Biotechnology Institute in Montreal (http://candida.bri.nrc.ca/candida/index.cfm?page=CaGeneSearch); Softberry protein data base (http://www.softberry.com/berry.phtml); ExPASy (http://expasy.hcuge.ch/index.html); and MatInd and MatInspector (17).

### Phenotypic Assay on L-Sorbose Medium

Either quantitative or spot dilution assays were carried out as previously described (7).

### Pulsed-Field Gel Electrophoresis (PFGE)

These techniques are described in references 3, 16, 19, 21 and 22.

### Fragmentation of Chromosome 5

Various fragmentation cassettes were made in pAK104 carrying URA3 as previously described (14). Also, a 0.5 kbp HindIII/SacI fragment carrying a C. albicans telomere was removed from pABTEL and inserted in pSFU1 containing URA3 flipper (12) that was digested with SacI/NotI. Different fragmentation cassettes were prepared in the resulting pAK197 by subcloning approximately 1 kbp long individual sequences of chromosome 5 or mapping sequences (MS) into a KpnI/XhoI site. Also, a 2.5 kbp XbaI fragment carrying the mutated IMH3 gene for resistance to mycophenolic acid (MPAR) was removed from pSFI1 and inserted in the XbaI site of pUC19. The resulting plasmid was linearized with SalI/PstI and ligated with a 0.5 SalI/PstI kbp fragment from pABTEL carrying a C. albicans telomere, thus, creating pAK195. A 1.0 kbp MS adjacent to fragmentation site 3 was amplified using genomic DNA of CAF4-2 and subcloned into pAK105 linearized with SacI/BamHI, thus creating pAK195, which was used to fragment chromosome 5 at site 3 in double constructs.

### Internal Deletion

Various deletion plasmids were made in pAK45 carrying URA3 as previously described (14). Also, various flanking sequences, either 0.5 kbp or 1.0 kbp length, were amplified by PCR from genomic DNA of CAF4-2 and subcloned into KpnI/XhoI and SacI/NotI sites of pSFU1 containing a URA3 flipper cassette, thus creating different deletion plasmids.

### Standard Verification of the Desired Genetic Change

The site of chromosomal integration of the deletion cassette was confirmed with two pairs of primers such that the PCR products were amplified across the 5′ and 3′ junctions, with one primer in each pair corresponding to a portion of the marker sequence. A single pair of primers amplifying across the end of the fragmentation cassette opposite to telomere was generated similarly. The eviction of the deletion cassette was confirmed using a pair of primers that amplifies across the eviction junction. Fragmented chromosomes or larger deletions were additionally visualized using PFGE (14).

### Approaches

In this work, the Sou⁻ strain CAF4-2 was used to identify CSU genes on chromosome 5. An initial screen for candidates was performed with chromosome 5 DNA library prepared in replicative plasmid pCA88. In this plasmid, a chromosome fragment was co-expressed with SOU1, which confers a Sou⁺ phenotype (9). When a plasmid insert carried a putative CSU gene for a negative regulator, SOU1 was repressed and the Sou⁻ phenotype of the strain CAF4-2 was restored. The final identification of the sequences that carry CSU genes came from deletions on chromosome 5, which shifted the phenotype from Sou⁻ to Sou⁺, similar to the loss of one homologue of chromosome 5. Independently, the phenotype shift from Sou⁻ to Sou⁺ was obtained due to chromosome 5 telomere-mediated fragmentation. The co-expression system was also used to analyze truncated or mutated portions of the original chromosome sequence.

### Multiple Deletions

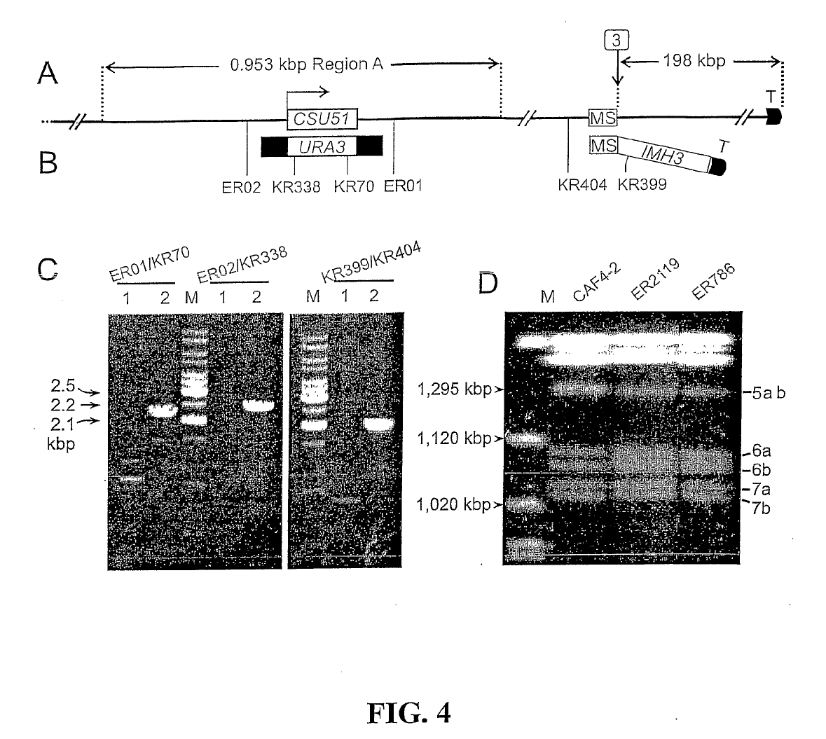

One means of deleting spatially separated sequences on a chromosome 5 was to remove, in one step, large portions of chromosome by telomere-mediated fragmentation. This method allows one to deliberately break a chromosome and is based on homologous recombination between so-called “mapping sequence” which is carried on chromosome and a plasmid. The plasmid also contains a selectable marker and C. albicans telomere, which stabilizes and allows propagation of one of two expected fragments (14, FIG. 4B). The sequences were also removed selectively, which required either two internal deletions or an internal deletion and a fragmentation. For two sequential manipulations, cells were transformed either with a URA3 flipper cassette (12) that was subsequently recycled or with two cassettes carrying URA3 and IMH3 for MPAR (13), respectively. The latter technique did not require an eviction step. It was irrelevant for our purposes whether one copy of the different sequences was sequentially deleted on the same or different chromosome 5 homologues.

### Confirmation Strategies

Relating manipulations with chromosome 5 and phenotypic changes depended upon the correct integration of deletion and fragmentation cassettes. We confirmed each site of cassette integration or eviction by PCR. The fragmented chromosomes and their sizes were additionally confirmed with PFGE as previously published (14). Only constructs with the desired alteration(s) and no other visible change in chromosome patterns, as confirmed by a full electro-karyotype of each construct, were finally assayed on L-sorbose plates. To ensure that the introduced genetic change confers a certain phenotype, up to 4 constructs of the same type were prepared independently.

Cloning Nine Candidate Sequences and Assigning Three of Them within the Approximately 395 kbp Terminal Portion in the Right Arm of Chromosome 5 Which Controls the Sou Phenotype

A chromosome 5 DNA library was prepared in vector pCA88 that carries SOU1 and that confers growth on sorbose, Sou⁺ (Approaches). A recipient Sou strain CAF4-2 was transformed with the library and 480 transformants obtained. A total of 9 plasmids that contained inserts denoted 133 through 135 and 137 through 142, were finally confirmed to repress SOU1 causing no growth on sorbose plates, and thus recovering the original Sou⁻ phenotype of CAF4-2.

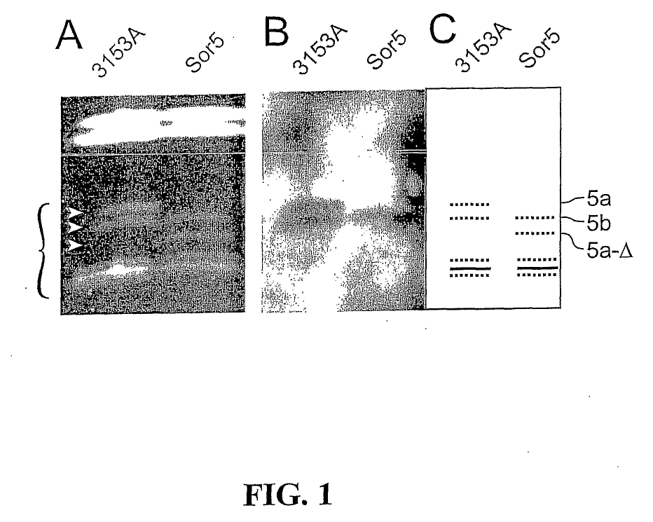

Further analysis included the exceptional Sou⁺ mutant Sor5 that was derived from C. albicans strain 3153A on an L-sorbose plate (5, 8). In contrast to the majority of Sou⁺ mutants, which lost one homologue of chromosome 5, Sor5 acquired a large deletion on homologue 5a, 5a-Δ, which presumably caused its phenotype (FIGS. 1A and C). According to Southern blot analysis, this deletion encompassed genes HIS1, ADH1, and CAG1 near the right telomere (FIG. 2A) (18). This result prompted the determination of a continuous sequence in the right arm of chromosome 5. Four contigs 19-10194 (contains telomere), 19-10171, 19-10093, and 19-2472 were aligned from assembly 19 of the C. albicans genomic sequence (Stanford Genome Technology Center), and the positions of two more contigs 19-10155 and 19-10105 (14) were determined. These contigs are designated as 1 to 6 (Table 1, FIG. 2A).

Six of nine cloned inserts, 133-135, and 140-142 were excised from the plasmids, used to prepare probes, which were hybridized to a chromosome blot of the mutant Sor5. As shown in FIG. 1B, two probes representing sequences 140 (6.892 kbp) and 135 (4.340 kbp), did not show signals with the truncated chromosome 5a-Δ, which suggested that these sequences map within the deletion. The other probes hybridized with both chromosomes 5b and 5a-Δ. The ends of all nine inserts 133-135 and 137-142 were sequenced and used to search assembly 19 of the Candida Genome. Sequence 140 mapped in contig 3 and 135 and an additional sequence 139 (4.547 kbp) mapped in contig 1 (FIG. 2C). Taken together with the loss of HIS1, ADH1, and CAG1, this result suggested that the Sor5 deletion extends to at least approximately 356 kbp, encompassing contigs 1 and 2 and portion of 3 (FIG. 2E). It also suggested that sequence 139 maps into deletion. Furthermore, as anticipated, the control terminal deletion of approximately 395 kbp between site 12 and right telomere (FIG. 2B) on one homologue of chromosome 5, conferred a Sou⁺ phenotype, confirming the results with the Sor5 mutant (Table 1) (see Approaches for the chromosome fragmentation). In contrast, another terminal deletion of approximately 88 kbp proximal to the left telomere (on another arm) did not change the phenotype. The remaining six sequences, including the four that were mapped outside of the Sor5 deletion, were distributed along chromosome 5 outside of the contigs 1-6. Importantly, each sequence was represented only once.

These results demonstrated that a terminal deletion of approximately 395-kbp on the right arm causes the Sou⁺ phenotype similar to the loss of one homologue of chromosome 5.

Identifying Functional Redundancy between Sequences 140 and 135 or 140 and 139.

Systematic truncation of one homologue of chromosome 5 showed that Sou⁻ and Sou⁺ phenotypes are generated according to the size of the removed portion. The larger truncations of approximately 345-378 kbp at sites 7-11 consistently caused a Sou⁺ phenotype, whereas strains with the shorter truncations at sites 3-6 remained Sou⁻ (FIG. 2B, Table 1). This result was consistent with finding that the approximately 395 kbp terminal portion of chromosome 5 was important for regulation (see above).

Unexpectedly, deletion of one copy of the intervening sequence of approximately 0.566 kbp between sites 6, Sou⁻, and 7, Sou⁺, which presumably contained CSU gene and which, remarkably, fell in the sequence 140 (FIGS. 2B and C), did not produce Sou⁺. Similarly, Sou⁺ was not produced by the two larger deletions that overlapped the intervening sequence and removed either one copy of 140, 6.892 kbp, or one copy of the extended 78.5 kbp sequence between fragmentation sites 4 and 9, the latter designated Deletion VII (FIG. 2D, Table 2). Also, the Sou⁻ phenotype of strain CAF4-2 did not change upon deletion of a single copy of either 135 or 139 (Table 2).

Furthermore, pairwise deletions revealed redundancy of regulatory elements. The Sou⁺ phenotype was produced upon concomitant removal of 140 and 135 or 140 and 139 (FIG. 2E, lines 1 and 2), but not 135 and 139 (see fragmentations in FIG. 2B and Table 2). Thus, all three sequences, 135, 139, and 140 are implicated in negative control of Sou phenotype representing independent functional Regions, but 135 and 139 are not redundant. Clearly, growth on sorbose requires a concomitant removal of particular Regions that are spatially separated on the right arm of chromosome 5.

Identifying a 0.953 kbp Functional Region A within Sequence 140

The sequence 140 was conveniently co-expressed with SOU1 on the vector pCA88 (see “Approaches”) and systematically truncated. The loss of a 0.953 kbp portion, which has been designated Region A, abolished the repressive properties of 140 and allowed SOU1 to confer growth on sorbose. Region A, which maps on the chromosome between sites 6 and 8 (FIG. 2F), overlaps with the previously determined intervening sequence of 0.566 kbp between sites 6 and 7 (see above). The function of Region A was corroborated by chromosome deletions (see below).

Confirming Region A and Identifying a Putative Functional Region B within the Remaining Portion of Sequence 140. Identifying an Additional Putative Functional Region C

The constructs of the type A-Δ 139-Δ became Sou⁺ similar to 140-Δ 139-Δ, which confirmed the above overexpression analysis and suggested that Region A contains CSU. On the other hand, the constructs A-Δ 135-Δ, against expectation and unlike the Sou⁺ constructs 140-Δ 135-Δ, did not become Sou⁺. This revealed that Region A is not equivalent to 140. The phenotypic differences are not due to the method for deleting the Regions, because the parallel constructs were similarly prepared, that is A-Δ 135-Δ and 140Δ 135-Δ combined internal deletions. On the other hand, A-Δ 139-Δ and 140-Δ 139-Δ combined internal deletion of A and 140 with terminal deletion at site 3 (for example, FIG. 2E, lines 1-3; Table 2). The inequality of A and 140 revealed at least one more Region, B, in the remaining portion of 140. It is clear that a combined deletion of A, B, and 135 confers growth; however it remains unclear whether deletion of both A and B are required or only deletion of B. Unlike a preliminary identification of Region A by co-expression of the corresponding portions of 140 with SOU1 on pCA88 (see above), Region B could not identified. The simple explanation is that splitting 140 outside Region A into two fragments destroyed the relevant CSU.

The analysis of Sou phenotype was extended due to concomitant deletion of A and 139 by combining internal deletion of A with terminal deletions at sites 1 and 2. It was found that combination with the shorter truncation at site 1 does not change Sou⁻ phenotype. However, in constructs with the truncation at site 2, which removed approximately 9 kbp longer portion, the Sou⁻ phenotype shifted to Sou⁺ similar to the constructs A-Δ 139-Δ containing fragmentation 3, as exemplified in FIG. 2E, line 3 (see also Table 2). This result revealed that there is at least one more functional Region, C, of 9 kbp, between sites 1 and 2.

Therefore, three Regions, 140, 135, and 139, were initially cloned from the library, which followed by confirmation using chromosome deletions. Two more Regions, B and C, were determined by chromosome deletions.

### Computational Analysis of Sequences 140, 135, 139, and C

Nucleotide sequences of all Regions were compared to the Candida genome databases, as well as the whole genome database of NCBI. One large ORF was identified in each of Regions A, B, and 139, and none of them was similar to any known sequence. Five ORFs were identified in Region C, two of them were not similar to any known sequence and the other three corresponded to the ASH2, CRH2, and PFK1 genes. In Region 135, two large ORFs were identified, YUH2 encoding a putative ubiquitin carboxyl-terminal hydrolase-like protein and SFC1 encoding a mitochondrial succinate-fumarate transporter. Their role in control of Sou phenotype remains to be seen. In this regard, there are examples of enzymes acting as regulators. Recently a mitochondrial enzyme Arg5,6, was shown to directly control gene expression (19).

### Identifying CSU51 in Region A

A 0.953 kbp of Region A was sequened and compared with the Stanford Genome Technology Center database of the Candida genome, which was 3 nucleotides shorter than our sequence. The NCBI ORF finder program indicated 5 putative ORFs, ORF1-ORF5, in Region A with the largest, ORF3, containing 255 bp (FIG. 2F). ORF2 and ORF3 were tandemly positioned with no space between them, so that the ORF3 stop codon is followed by the ORF2 start codon. ORF3 and ORF4 completely overlap. Interestingly, the first fragmentation site, 7, that removed a portion of chromosome 5 large enough to produce growth on sorbose, cut ORF3 in the middle, removing the upstream region of ORF4 and removing ORF2.

In order to identify the ORF that is responsible for the repressive properties of Region A, this Region carrying one or another of the five ORFs destroyed by frameshift mutation introduced by site directed mutagenesis, as indicated by asterisks (FIG. 2F), was individually subcloned in vector pCA88, thus, creating five replicative plasmids. Care was taken to introduce a mutation in ORF4 such that only an innocuous base pair was introduced in ORF3. Mutated ORFs were sequenced in order to confirm the mutation. The five plasmids were independently transformed in the Sou⁻ strain CAF4-2, and transformed cells were plated on sorbose medium in order to co-express the altered Regions A with SOU1 (FIG. 2G). It was determined that only destruction of ORF3, but not the other putative ORFs, abolished the function of Region A, de-repressed SOU1, and allowed growth on sorbose.

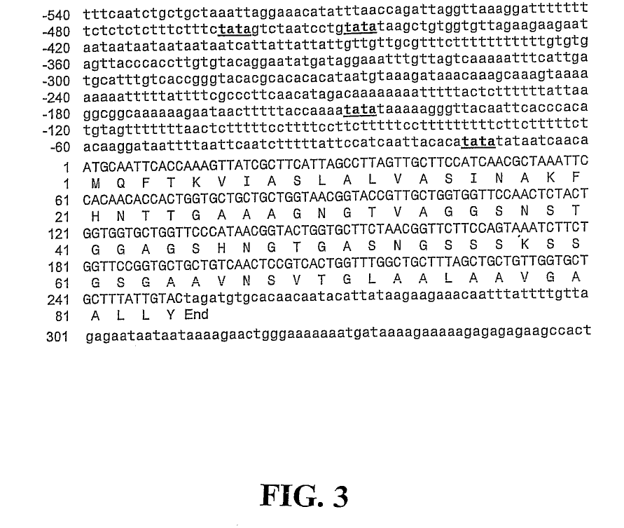

RT-PCR was used to determine whether ORF3 is expressed in strain CAF4-2 with the pair of primers, one of which contained oligodT. The product of 398 bp was amplified. The sequencing revealed that 255 bp represented ORF3 and 54 bp represented the 3′ end of the transcript of ORF3 or 3′ UTR (FIG. 3). This result showed that ORF3 is expressed, and also confirmed that ORF3 does not extend to ORF2.

One copy of ORF3 was deleted, followed by chromosome fragmentation at site 3 (FIG. 4), thus resulting in the construct of the type orf3-Δ fragmentation 3 (C-Δ 139-Δ). This construct became Sou⁺, similar to removal of either 140 or Region A combined with the loss of C and 139 due to fragmentation 3 (FIG. 2E, line 4).

In an additional experiment we obtained the data, which supported the results with deletions on the chromosome. Region A was systematically truncated on plasmid pCA88 similar to this in section “Identifying a 0.953 kbp Functional Region A within Sequence 140”, and it was found that the repressive properties of Region A are due to the presence of ORF3.

Taken together, the present invention provides a CSU gene on chromosome 5, which was denoted CSU51. The sequence for CSU51 has been deposited in the GenBank database under Accession No. DQ068774. The nucleic acid sequences, polypeptide sequences and information set forth under GenBank Accession No. DQ068774 are incorporated herein by this reference. Also provided herein are CSU52, CSU53 and CSU55 which, like CSU51 are relatively small polypeptides can function as transcriptional regulators.

### CSU51 is Not an Essential Gene

A single copy of CSU51 was deleted in Sou⁺ mutant Sor19 containing one copy of chromosome 5. The deletion was done similarly to the deletion of CSU51 (ORF3) in construct orf3-Δ fragmentation 3 and also similarly confirmed by PCR in three independent transformants (see above). Also, PCR failed to amplify the coding region of CSU51 with primers KR360/KR361 (Table 3 of Supporting Information) in those transformants. This suggests that CSU51 is not an essential gene. Strains csu51-Δ/csu51-Δ were Sou⁺ like the parental strain Sor19 (CSU51-Δ/csu51-Δ).

### Sequence Analysis of CSU51

The nucleotide sequence of the 255 bp of CSU51 encodes a deduced protein of 84 amino acids with a proposed molecular mass of 9.24 kD and a high content of serine, 16%, alanine, 21%, and glycine, 15% (FIG. 3). ExPASy tools predicted that Csu51p has helix-loop-helix (HLH) structure with helix 1 and helix 2 spanning the regions between amino acids 2-3 to 17-20 and 63-67 to 82-84, respectively. However, there is no significant similarity between helix 1 and 2 regions of Csu51p and other HLH proteins (20). Csu51p apparently localizes to the nucleus according to the Softberry protein database, which implies that Csu51p might be a transcription factor.

Five separate Regions, A, B, C, 135, and 139 were discovered within an approximately 209 kbp of the right arm of chromosome 5 that are functionally, but not structurally, redundant. These Regions repress the growth on sorbose medium presumably via repression of a metabolic gene SOU1 on another chromosome. These five Regions may belong to two redundant regulatory pathways, one including A and B and another including 135, C, and 139. Clearly, hemizygous deletion of the following did not produce Sou⁺ phenotype: any single Region; the pair of Regions A and B; the three Regions 135, C, and 139. However, Sou⁻ strains became Sou⁺ due to hemizygous deletion of all five Regions or due to hemizygous deletions that combined the Regions from two putative pathways: A, B, and 135; A, 135, C, and 139; A, C, and 139. A weak CSU on chromosome 1, previously reported by this group, an essential gene BMH1, deletion of one copy of which increased SOU1 transcript by two-fold (10). In addition, three other potential weak regulators, CSU2, CSU5, and CSU6, from different chromosomes, were partially characterized. The complexity and redundancy of the negative regulation could reflect a general redundancy of metabolic and regulatory pathways in cell, which now becomes increasingly obvious (21, 22).

If each Region encompasses at least one CSU gene, CSU51-CSU55, then three more novel genes in Regions B, C, and 139 are expected, similar to novel CSU51, which resides in Region A. Most informative in this respect is the fact that each of nine fragments of chromosome 5, 133-135 and 137-142 that were cloned from a chromosome 5 DNA library was represented only once. This is especially important because of the small size of the library versus a relatively large number of cloned fragments. It is possible that all or some of these fragments also contain CSUs. Furthermore, some additional CSUs could be uncovered in still unidentified portions of the chromosome including inside of the right arm. One explanation for the growth on sorbose due to the loss of one copy of CSUs carried in Regions A, B, C, 135, and 139, but not all CSUs, could be that these genes represent all redundant pathways.

Multiple redundant regulators explain in a simple elegant way why the entire chromosome needs to be lost in Sou⁺ mutants. It remains to be seen if the chromosomes 4 and 6, which are lost in Flu^{R} and Aru⁺ mutants, respectively, also contain multiple negative regulatory genes. These findings would imply that multiple groups of C. albicans genes are distributed nonrandomly among the different chromosomes. The diminution of the dose of multiple negative regulatory genes via the loss of an entire chromosome 5 of C. albicans appears to involve the same mechanism that produces the diminution of the dose of multiple tumor suppressor genes in humans. For example, bladder cancer can arise by the loss of chromosome 9, which contains tumor suppressors in at least four spatially separated regions (23).

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Bennett, R. J. & Johnson, A. D. (2003) EMBO J. 22, 2505-2515.
2. Rustchenko, E. & Sherman, F. (2002) in Fungi Pathogenic for Humans and Animals, ed. Howard, D. H. (New York: Marcel Dekker Inc.), pp. 723-776.
3. Janbon, G., Sherman, F. & Rustchenko, E. (1998) Proc. Natl. Acad. Sci. U.S.A. 95, 5150-5155.
4. Janbon, G., Sherman, F. & Rustchenko, E. (1999) Genetics 153, 653-664.
5. Rustchenko, E. P., Howard, D. H. & Sherman, F. (1994) J. Bacteriol. 17, 32311-3241.
6. Perepnikhatka, V., Fischer, F. J., Niimi, M., Baker, R. A., Cannon, R. D., Wang, Y.-K., Sherman, F. & Rustchenko, E. (1999) J. Bacteriol. 181, 4041-4049.
7. Wellington, M. & Rustchenko, E. (2005) Yeast 22, 57-70.
8. Rustchenko, E. (2003) in Recent Research developments in Bacteriology, ed. Pandalai, S. G. (Transworld Research Network), pp. 91-102.
9. Wang, Y.-K., Das, B., Huber, D. H., Wellington, M., Kabir, M. A., Sherman F. & Rustchenko, E. (2004) Yeast 21, 685-702.
10. Greenberg, J. R., Price, N. P., Oliver, R. P., Sherman, F, & Rustchenko, E. (2005) Yeast (In press).
11. Fonzi, W. A. & Irwine, M. Y. (1993) Genetics 134, 717-728.
12. Morschhauser, J., Michel, S. & Staib, R. (1999) Mol. Microbiol. 32, 547-556.
13. Staib, P., Iretschmar, M., Nichterlein, T., Köhler, G., Sonja, M., H of, H., Hacker, J. & Morschhauser, J. (1999) Mol. Microbiol. 32, 533-546.
14. Kabir, M. A. & Rustchenko, E. (2005) Gene 345, 279-287.
15. Cannon, R. D., Jenkinson, H. F., Shepherd, M. G. (1992) Mol. Gen. Genet. 235, 453-457.
16. Rustchenko-Bulgac, E. P. (1991) J. Bacteriol. 173, 6586-6596.
17. Quandt, K., Frech, K., Karas, H., Wingender, E. & Werner, T. (1995) Nucleic Acids Res. 23, 4878-4884.
18. Selmecki, A., Bergmann, S. & Berman, J. (2005) Mol. Microbiol. 55, 1553-1565.
19. Hall, D. A., Zhu, H., Zhu, X., Royce, T., Gerstein, M. & Snyder, M. (2004) Science 306, 482-484.
20. Chavali, G. B., Vijayalakshmi, C. & Salunke, D. M. (2001) Proteins 42, 471-480.
21. Krakaur, D.C. & Plotkin, J. B. (2002) Proc. Natl. Acad. Sci. U.S.A. 99, 1405-1409.
22. Normanly, J. & Bartel, B. (1999) Current Opin. Plant Biol. 2, 207-213.
23. Williams, S. V., Sibley, K. D., Davies, A. M., Nishiyama H., Hornigold, N., Coulter, J., Kennedy, W. J., Skilleter, A., Habuchi, T., Knowles, M. A. (2002) Genes Chromosomes Cancer 34, 86-96.
24. Huber, D. H. & Rustchenko, E. (2001) Yeast 18, 261-272.
25. Golemis, E. A., Gyuris, J., Brent, R. (2003) in Current protocols in molecular biology, eds. Ausubel, F. M., Brent, R., Kingston, R. E., Moore D. D., Sidman, J. G., Smith, J. A. & Struhl, (John Wiley and Sons, New York, N.Y.), pp. 13.0.1-13.14.17.

TABLE 1
Sou phenotype* of strains in which one chromosome 5 is fragmented at various sites

| | Fragment. site† | | | | | | | | | |
|---|---|---|---|---|---|---|---|---|---|---|
| | 12 | 11 | 10 | 9 | 8 | 7 | 6 | 5 | 4 | 3 |
| % Sou⁺ | 34; 57 | 16; 34; 37; | 82; 68; | 59; 61 | 52; 42 | 62 | 0.0 | 0.2; 1.8 | 0.0; 0.0; | 0.4; 0.6 |
| colonies | | 53; 85 | 42 | | | | | | 0.2 | |

*The Sou phenotype was determined by plating approximately 500 and 5,000 cfu on L-sorbose plates in duplicate, as well as approximately 500 cfu on SD plates in duplicate, and measured as percent colonies on L-sorbose plate compared to colonies on SD plate. The Sou⁺ phenotype and the corresponding sites are indicated in bold.
†The phenotypes for fragmentation at sites 1 and 2 were determined in combination with deletion of Region A (see Table 2). See Table 4 of the Supporting Information for the site positions, as well as for the values of the control strains.

TABLE 2
Sou phenotype* of Ura⁺ and Ura⁻ constructs lacking
one copy of various Regions 140 (A and B), A, 135, C
or 139 on chromosome 5

| Abbreviated genotype | % Sou⁺ colonies |
|---|---|
| Sou⁺ hemizygous deletants† | |
| 140-Δ 135-Δ | 16; 12; 17 |
| Deletion VII (140-Δ) fragmentation 3 (C-Δ 139-Δ) | 43; 27 |
| A-Δ fragmentation 2 or 3 (C-Δ 139-Δ) | 73; 66; 70; 32; |
| | 78; 49 |
| csu51-Δ fragmentation 3 (C-Δ 139-Δ) | 76; 66; 40; 72 |
| For fragmentations 7, 8 (A-Δ 135-Δ C-Δ 139-Δ), | |
| and 9-12 (140-Δ 135-Δ C-Δ 139), see Table 1 | |
| Sou⁻ hemizygous deletants | |
| 140-Δ | 0.4; 0.1; 0.2 |
| Deletion VII (140-Δ) | 0.1; 0.1; 0.1; 0.1 |
| 135-Δ | 0.0; 0.0; 0.0; 0.2 |
| csu51-Δ | 0.1; 1.3; 0.7; 0.3 |
| A-Δ | 2.1; 3.7; 3.8 |
| A-Δ 135-Δ | 0.2; 0.6; 0.8 |
| A-Δ C-Δ | 0.4; 0.4; 3.1 |
| A-Δ fragmentation 1 (139-Δ) | 0.2; 0.4 |
| For fragmentations 3 (C-Δ 139-Δ) and 4-6 | |
| (135-Δ C-Δ 139-Δ) see Table 1, FIGS. 2B and C | |
| Sou⁻ control strains | |
| Ura⁻ Sou⁻ original CAF4-2 | 0.8; 0.8 |
| Ura⁺ Sou⁻ derivative of CAF4-2, ER305^{‡} | 0.4; 0.1; 0.2; 0.1 |
| Sou⁺ control strain | |
| Ura⁻ Sou⁺ mutant Sor19 lacking one chromosome 5 | 67; 61; 79 |

*For the explanation see Table 1. The Sou⁺ phenotype is indicated in bold.
†One copy of the indicated Region or gene is removed by either by internal deletion or chromosome fragmentation as indicated. Multiple values designate independent constructs, which are listed in footnote. Construct csu51-Δ fragmentation 3 (C-Δ 139-Δ) represented by a single strain ER2119 was repeatedly assayed on sorbose plates. The chromosome 5 in all constructs is represented by either two homologues of regular length or by one regular and one truncated homologue, as analyzed with PFGE.
^{‡}Integrative plasmid pRC3915 (LEU2 URA3) inserted at LEU2 locus in strain CAF4-2. The following strains (abbreviated genotypes) are presented in this table: ER1705, ER1792, ER1793 (140-Δ 135-Δ); ER1291, ER1292 [Deletion VII (140-Δ) fragmentation 3 (C-Δ 139-Δ)]; ER1463, ER1606, ER1644 [A-Δ fragmentation 3 (C-Δ 139-Δ)]; ER1826, ER1827, ER1833 [A-Δ fragmentation 2 (C-Δ 139-Δ)]; ER2119 [csu51-Δ fragmentation 3 (C-Δ 139-Δ)]; ER469, ER470, ER472 (140-Δ); ER1266, ER1271, ER1276, ER1277 [Deletion VII (140-Δ)]; ER514, ER515, ER517, ER518 (135-Δ); ER2016, ER2017, ER2108, ER2112 (csu51-Δ); ER 1314, ER1313, ER1312 (A-Δ); ER1968, ER1970, ER1971 (A-Δ 135-Δ); ER1963, ER1964, ER1965 (A-Δ C-Δ); ER1484, ER1485 [A-Δ fragmentation 1 (139-Δ)].

TABLE 3

Primers used in this study

| | | |
|---|---|---|
| Name | Sequence (5′→3′) | Purpose |

| | |
|---|---|
| | Site of |
| Chromosome fragmentation | fragmentation |

| | | |
|---|---|---|
| KR218 | TCG GGT ACC ATT TAC AAA AAT TCA ATA TCC AAG | 1 |
| | KpnI | |
| KR219 | TCG CTC GAG GTT GTT CCA GAT ACT AAC TAA GTC | |
| | XhoI | |
| KR307 | TCG GGT ACC TCA GCA GTT GAA ACA TCT TTT | 2 |
| | KpnI | |
| KR308 | TCG CTC GAG CAC AAC TGT TTC TTA CCA ACA | |
| | XhoI | |
| KR43 | GGG GGC TCG GAG CTC GGC AAT TTG TAA ACC TAA ACA | 3 |
| | SacI | |
| KR44 | GGG GGC TCG GGA TCC ACC ATG TCT CCA TTA GAA CAT | |
| | BamHI | |
| KR139 | GGG GGC TCG GGA TCC TCC TAT CCT ATC CTA TCT CAC | 5 |
| | BamHI | |
| KR140 | GGG GGC TCG GAG CTC ATG CAA CAT ATA TAT AAC TAT | |
| | SacI | |
| KR149 | GGG GGC TCG GGA TCC TAT GTG TAT AAT ATA TAA AAA | 6 |
| | BamHI | |
| KR150 | GGG GGC TCG GAG CTC ATT TCA TTG ATG CAT TTG TCA | |
| | SacI | |
| KR151 | GGG GGC TCG GGA TCC TAC CGT TAC CAG CAG CAG CAC | 7 |
| | BamHI | |
| KR152 | GGG GGC TCG GAG CTC CTA AAA TCA TCA TAA CCA GAG | |
| | SacI | |
| KR153 | GGG GGC TCG GGA TCC AAC AAA CCT TTG TTA CAT TGT | 8 |
| | BamHI | |
| KR154 | GGG GGC TCG GAG CTC TTG TTT GGT GTG AAA GAT GCT | |
| SacI | | |
| KR107 | GGG GGC TCG GGA TCC CGG TGC AAC AAT TTT TAT TAA | 10 |
| | BamHI | |
| KR116 | GGG GGC TCG GAG CTC TTG TTG GTT TTG TTA CTA TTT | |
| | SacI | |
| KR73 | GGG GGC TCG GAG CTC CAT AGA TAT GAA TAT CTC AGC | 13 |
| | SacI | |
| KR74 | GGG GGC TCG GGA TCC TTC CTC GTC ATT AAA CCG AAA | |
| | BamHI | |

| | |
|---|---|
| Frameshift mutation in Region A | ORF name |

| | | |
|---|---|---|
| KR455 | ATT ATG AAA AAA AAG TAA AGA AGA ACA CAA CAT TCA AAT T | 1 |
| KR456 | AAT TTG AAT GTT GTG TTC TTC TTT ACT TTT TTT TCA TAA T | |
| KR445 | TAC ATT ATA AGA AGA AAC AAT TTA TTT TGT TAG AGA ATA CAT AAT | 2 |
| KR05 | GGG GGC TCG GGA TCC ATA TCA TTT CTT CCC ATA | |
| KR279 | AAC GGT ACC GTT ACC AGC AGC AGC ACC AGT GGT GTT GTG GAA TTT | 3 |
| | AGC GTT GAT GGA AGC AAC TAA GGC TAA TTG AAG CG | |
| KR06 | GGG GGC TCG CTG CAG TCA AAA ATT TCA TTG ATG | |
| KR435 | GGT AAC GGT ACC GTT GCT GGT GGT TCC AAC TCT ACT GGT GGT GCT | 4 |
| | GGT TCA CAT AAC GGT | |
| KR05 | (See above) | |
| KR434 | TCG CTG CAG TCA AAA ATT TCA TTG ATG CAT TTG TCA CCG GGT | 5 |
| | ACA CGC ACA CAC ATA ATG TAA AGA TAA ACA AAG CAA AGT AAA | |
| | AAA AAC ATT TTT ATT | |
| KR05 | (See above) | |

| | |
|---|---|
| Deletion of Region or gene | Region/Gene |

| | | |
|---|---|---|
| KR376 | TCG GAG CTC AAC ACA CAC AAG ATG TTT AAC | CSU51 |
| | SacI | |
| KR377 | TCG GCG GCC GC A TGT GCA CAA CAA TAC ATT AT | |
| | NotI | |
| KR378 | TCG CTC GAG TGT TGA TTA TAT ATA TGT GTA | |
| | XhoI | |
| KR379 | TCG GGT ACC ATA AAA TAC CAC CAC AAC AAG | |
| | KpnI | |
| KR226 | TCG GGT ACC TAA AAA ACT TCA AGA TAT TTT CAA | A |
| | KpnI | |
| KR227 | TCG CTC GAG TTA ATA ATA ATA AAG TAG TTT GAA | |
| | XhoI | |
| KR228 | TCG GCG GCC GC C TAA CAA ATT TCC TAT CAT ATT CC | |
| | NotI | |
| KR229 | TCG GAG CTC TGT ATT ATT GTA TTG TGT AAA AAT | |
| | SacI | |
| KR15 | GGG GGC TCG GAG CTC AGG GTG AAA TAT TGT GAC GGA | CA135 |
| | SacI | |
| KR16 | GGG GGC TCG GGA TCC TCT GGG TGC ACA TCA GTA ACG | |
| | BamHI | |
| KR17 | GGG GGC TCG CTG CAG CAA ATG AGA ACA AAC CGT GTC | |
| | PstI | |
| KR18 | GGG GGC TCG AAG CTT GGT TAT CGG AAC ATT GT TCG | |
| | HindIII | |
| KR19 | GGG GGC TCG GAG CTC ACT GTA ATG AAT GAG TTA AGG | CA140 |
| | SacI | |
| KR20 | GGG GGC TCG GGA TCC CAT AAT TGT TAA TTC ATT ATT | |
| | BamHI | |
| KR21 | GGG GGC TCG CTG CAG ATA ATC AAG ATC AAA TAC AAT | |
| | PstI | |
| KR22 | GGG GGC TCG AAG CTT TGA TAA TGA TGG TGA GGG AGA | |
| | HindIII | |
| KR15 | (See above) | CA135 |
| | | (URA3 flipper) |
| KR286 | TCG GCG GCC GC T CTT GGT GCA CAT CAG TAA CG | |
| | NotI | |
| KR287 | TCG CTC GAG CAA ATG AGA ACA AAC CGT GTC | |
| | XhoI | |
| KR288 | TCG GGT ACC GGT TAT CGG AAC ATT GTT TCG | |
| | KpnI | |
| KR19 | (See above) | CA140 |
| | | (URA3 flipper) |
| KR300 | TCG GCG GCC GC C ATA ATT GT AAT TCA TTA TT | |
| | NotI | |
| KR301 | TCG CTC GAG ATA ATC AAG ATC AAA TAC AAT | |
| | XhoI | |
| KR302 | TCG GGT ACC TGA TAA TGA TGG TGA GGG AGA | |
| | KpnI | |
| KR141 | GGG GGC TCG GGA TCC TAA TGA ATT TGA ATT TTC TCA | VII |
| | BamHI | |
| KR142 | GGG GGC TCG GAG CTC TGG ATC ATC AAT TAA TAT AGT | |
| | SacI | |
| KR113 | GGG GGC TCG CTG CAG ACA ACC TAC CAC AAC TGA ACA | |
| | PstI | |
| KR114 | GGG GGC TCG AAG CTT AAT TGA AAT ACA AAA ATC TAT | |
| | HindIII | |

Junction of Region or gene deletion

| | | |
|---|---|---|
| ER01 | ACT GTA ATG AAT CAG TTA AGG | CSU51 |
| KR70 | CAG TTG AAG AAA GAA ATA GAA | |
| ER02 | CAA ACT GCA AAC TGT TTT TAA | |
| KR338 | AAT GGT GAT GTC TAG TGG GTT | |
| ER01/KR70, | (See above) | A |
| ER02/KR338 | | |
| ER01/KR70, | (See above) | CA140 |
| KR142 | | |
| KR71 | TTA GTG TTG ACT GTC ATA TCT | |
| KR338/KR70, | (See above) | CA135 |
| RR71 | | |
| KR96 | ATA GTT CAT AAC ACT TTG TTT AGA | |
| KR97 | GCT GTA CGT GTA CCT AAC AAA AAA | |

| | |
|---|---|
| | Site of |
| Confirmation of chromosome fragmentation | fragmentation |

| | | |
|---|---|---|
| KR399 | CAG CTG AAC TAG GGA AAT TGA | 3 |
| KR404 | TCT ATG TCT GTG TCT TTC GAT AAC | |
| KR70/ER02, | (See above) | 5-7 |
| KR338 | (See above) | 2 |
| KR372 | TTA CCA TGT AAA TCC AAC ACT | |
| Sequencing | | |
| KR350 | TAG TAG TAC TGG TGA TGA TCA | |
| KR351 | CAA TCT GCT GCT AAA TTA GGA | |
| KR352 | ACT AGT AGC ATT AGT CTC AGA | |

Also used M13 forward (−41) and M13 reverse (−27) as previously described (Kabir and Rustchenko, 2005).

TABLE 4
Sou phenotype* of strains in which one chromosome 5 is fragmented at
various sites, as well as of control strains with no chromosome 5 fragmentation†

| | | | Strains- | Strains- | |
|---|---|---|---|---|---|
| Contig | | Distance | with chr. 5 | with no | |
| Name | | (kb) from | fragmentation | fragmentation | |
| This work | Fragmentation | right | (% Sou⁺ | (% Sou⁺ | Deleted |
| (Assembly 19) | site | telomere | colonies) | colonies) | Region |
| 1 (19-10194) | 1‡ | 147 | See Table 2 | See Table 2 | 139 |
| 1 (19-10194) | 2‡ | 156 | See Table 2 | See Table 2 | 139, C |
| 1 (19-10194) | 3 | 196 | 0.4; 0.6 | 0.6 | 139, C |
| 1 (19-10194) | 4 | 273 | 0.0; 0.0; 0.2 | 0.1 | 139, C, |
| | | | | | 135 |
| 3 (19-10093) | 5 | 343 | 0.2; 1.8 | 0.0 | 139, C, |
| | | | | | 135 |
| 3 (19-10093) | 6 | 344 | 0.0 | 0.0; 0.0; 0.1; 0.1; | 139, C, |
| | | | | 0.0; 0.0; 0.1 | 135 |
| 3 (19-10093) | 7 | 345 | 62 | ND | 139,C |
| | | | | | 135, A |
| 3 (19-10093) | 8 | 346 | 52; 42 | 2.1; 0.2 | 139, C |
| | | | | | 135, A |
| 3 (19-10093) | 9 | 351 | 59; 61 | 3.7; 0.0; 0.1; | All |
| | | | | 0.1; 0.0; 0.0; | |
| 3 (19-10093) | 10 | 370 | 82; 68; 42 | ND | All |
| 3 (19-10093) | 11 | 378 | 16; 34; 37; 53; 85 | 0.1; 0.6; 0.1 | All |
| 4 (19-2472) | 12 | 395 | 34; 57 | 0.2; 0.1 | All |

*For the explanation see Table 1. The Sou⁺ phenotype is indicated in bold.
†Transformants from the same transformation plate lacking uridine either contained or did not contain fragmented chromosome 5. As we have previously reported, the success of chromosome fragmentation is always less than 100% and varies at different sites (14). Supposedly, Ura⁺ transformants with the intact chromosome 5 have incorporated fragmentation cassette somewhere else in genome. These can be used as control for Sou phenotype.
‡Fragmentation was performed in combination with deletion of Region A.
ND Not determined.
Note that presented here are only Sou phenotypes of transformants containing either solely chromosome 5 fragmentation or none. Transformants containing undesirable alteration(s) due to mutagenesis were not considered.

The following strains (site of fragmentation) are presented in this table: ER784, ER786 (3); ER928, ER929, ER931 (4); ER950, ER958 (5); ER1082 (6); ER1443 (7); ER1092, ER1114 (8), ER978, ER980 (9); ER896, ER900 (10); ER901, ER904, ER905, ER906, ER907 (11); ER503, ER506 (12).

### CSU Sequences

SEQ ID NO: 1
M Q F T K V I A S L A L V A S I N A K F
H N T T G A A A G N G T V A G G S N S T
G G A G S H N G T G A S N G S S S K S S
G S G A A V N S V T G L A A L A A V G A
A L L Y
SEQ ID No: 2
ATGCAATTCACCAAAGTTATCGCTTCATTAGCCTTAGTTGCTTCCATCAACGCTAAATTC
CACAACACCACTGGTGCTGCTGCTGGTAACGGTACCGTTGCTGGTGGTTCCAACTCTACT
GGTGGTGCTGGTTCCCATAACGGTACTGGTGCTTCTAACGGTTCTTCCAGTAAATCTTCT
GGTTCCGGTGCTGCTGTCAACTCCGTCACTGGTTTGGCTGCTTTAGCTGCTGTTGGTGCT
GCTTTATTGTAC
SEQ ID NO: 3
tttcaatctgctgctaaattaggaaacatatttaaccagattaggttaaaggattttttt
tctctctctttctttctatagtctaatcctgtatataagctgtggtgttagaagaagaat
aataataataataataatcattattattattgttgttgcgtttctttttttttttgtgtg
agttacccaccttgtgtacaggaatatgataggaaatttgttagtcaaaaatttcattga
tgcatttgtcaccgggtacacgcacacacataatgtaaagataaacaaagcaaagtaaaa
aaaaatttttattttcgcccttcaacatagacaaaaaaaatttttactcttttttattaa
ggcggcaaaaaagaataactttttaccaaaatatataaaaagggttacaattcacccaca
tgtagtttttttaactctttttccttttccttctttttcctttttttttcttctttttct
acaaggataattttaattcaatctttttattccatcaattacacatatatataatcaaca
ATGCAATTCACCAAAGTTATCGCTTCATTAGCCTTAGTTGCTTCCATCAACGCTAAATTC
CACAACACCACTGGTGCTGCTGCTGGTAACGGTACCGTTGCTGGTGGTTCCAACTCTACT
GGTGGTGCTGGTTCCCATAACGGTACTGGTGCTTCTAACGGTTCTTCCAGTAAATCTTCT
GGTTCCGGTGCTGCTGTCAACTCCGTCACTGGTTTGGCTGCTTTAGCTGCTGTTGGTGCT
GCTTTATTGTACtagatgtgcacaacaatacattataagaagaaacaatttattttgtta
Gagaataataataaaagaactgggaaaaaaatgataaaagaaaaagagagagaagccact
SEQ ID NO: 4
CAA TTC ACC AAA GTT ATC GCT
SEQ ID NO: 5
GTA CAA TAA AGC AGC ACC AAC
SEQ ID NO: 6
Amino acid sequence of CSU52
M C I Y L L H I V M M L Q N F I I T V N K F F V F A P P V N L L F C Q
T G I L V F H H H H Q T R A N N T T I L G R D R I I T R R
SEQ ID NO: 7
Amino acid sequence of CSU53
M S S T Q K Q K K G A V D F V A G G V A G L F E A L C C H P L D T I K
V R M Q L Y K K S G Q K P P G F I K T G V N I V Q K E G F L S L Y K G
L G A V V I G I V P K M A I R F S S Y E F Y R S F F L D E N G K I S T
G K T F L A G V G A G I T E S V M V V N P M E V V K I R L Q A Q H H S
M K D P L D I P K Y R N A P H A A Y L I V K E E G F S T L Y R G V S L
T C A R Q A T N Q G A N F A T Y S T I K A Y L Q K Q Q N T E L L F A W
Q T S I V G L I S G A V G P L T N A P L D T I K T R L Q K S K F T N K
E N G L V R I V K I G K Q L V K E E G I N A L Y K G I T P R I M R V A
P G Q A V V F T V Y E A V K H Y L T N E P T A
SEQ ID NO: 8
Amino acid sequence of CSU55
M I L C P T S Y V W Y V K Q L V I D N F F E I M Y N L V K S W S L I
K K K R M K
SEQ ID NO: 9
Nucleic acid sequence encoding CSU52
atgtgcatat atttattaca tattgtaatg atgttacaga attttattat taccgtgaat
aaattttttg tttttgcacc ccctgtaaat ttgttatttt gtcaaactgg tattcttgtg
ttccaccacc accaccagac ccgtgctaat aatactacca ttttagggcg agacagaatt
ataacaagac gataa
SEQ ID NO: 10
Nucleic acid sequence encoding CSU53
atgtcttcta cacaaaaaca aaagaagggt gctgttgatt tcgttgctgg gggtgttgct
ggtttattcg aagctttgtg ttgtcatcca ttagatacca tcaaagtgag aatgcaatta
tacaaaaaat ccggtcaaaa accaccaggt tttattaaaa caggtgtcaa tattgtccaa
aaagaaggat ttttgtcatt atataaaggt ttgggggccg ttgttattgg tattgtgcca
aaaatggcta tcagattcag ttcatatgaa ttttaccgtt cattcttttt agacgaaaat
ggcaaaattt ccactggtaa gactttcctt gctggtgttg gtgctggtat taccgaatct
gtcatggttg ttaatcctat ggaagttgtg aaaattagat tacaagcaca acatcattct
atgaaggacc cattggacat tccaaaatac agaaacgctc ctcatgctgc atatcttatt
gtcaaggaag aaggtttcag tactttatac cgtggtgttt ctttaacttg tgccagacaa
gctaccaacc aaggtgctaa ctttgctaca tattctacca tcaaagcata tcttcaaaaa
caacaaaaca ctgaattatt accagcatgg caaaccagta ttgtcggttt gatttctggt
gcagtcggtc cattaaccaa tgctccattg gataccatta aaacaagatt acaaaagagt
aagtttacca acaaggaaaa cggattggtt cgtattgtca aaatcggtaa acaattagtc
aaagaagaag gtattaacgc tttgtacaag ggtatcactc caagaatcat gagagttgct
ccaggtcaag ctgtggtatt cacagtgtat gaagctgtca aacattattt gacaaatgaa
cctactgctt aa
SEQ ID NO: 11
Nucleic acid sequence encoding CSU55
atgattttat gccctacatc gtacgtttgg tacgtgaaac aacttgtaat tgataatttt
tttgaaatta tgtataattt agttaaaagt tggagtttga tcaagaaaaa gcgaatgaag
tga

1. A purified polypeptide comprising an amino acid sequence at least about 95% identical to the sequence of SEQ ID NO: 6.

2. The polypeptide of claim 1 comprising a CSU52 polypeptide comprising SEQ ID NO: 6.

3. The polypeptide of claim 2 with one or more conservative amino acid substitutions.

4. A nucleic acid encoding the polypeptide of claim 2.

5. The nucleic acid of claim 4, wherein the nucleic acid comprises SEQ ID NO: 9.

6-10. (canceled)

11. A Candida albicans cell comprising the nucleic acid of claim 5.

12. An isolated antibody or fragment thereof that specifically binds the polypeptide of claim 2.

13-16. (canceled)

17. A method for detecting the presence of Candida albicans in a sample, comprising the steps of:
(a) contacting a biological sample with at least two oligonucleotide primers, each primer consisting of 10 to 200 contiguous nucleotides of the nucleic acid of claim 5 or the complement thereof, in a reverse transcriptase polymerase chain reaction; and
(b) detecting in the sample a polynucleotide sequence that amplifies in the presence of said oligonucleotide primers, wherein the presence of an amplified polynucleotide sequence indicates the presence of Candida albicans in the sample.

18. A polynucleotide probe comprising a polynucleotide at least 25 contiguous nucleotides of the nucleic acid of claim 5.

19. A method for detecting the presence of Candida albicans in a sample, comprising the steps of:
(a) contacting a biological sample with the probe of claim 18 under conditions that allow the probe to selectively bind a CSU52 nucleic acid; and
(b) detecting the presence of a CSU52 nucleic acid, whereby the presence a CSU52 nucleic acid indicates the presence of Candida albicans in the sample.

20. A method of detecting the presence of Candida albicans in a sample comprising:
a) contacting a sample with an antibody to the polypeptide of claim 1; and
b) detecting the antibody bound to the CSU52 polypeptide in the sample, wherein binding of CSU52 polypeptide to the antibody indicates the presence of Candida albicans in the sample.

21. A Candida albicans cell comprising the CSU52 nucleic acid sequence of claim 4 wherein the nucleic acid sequence is mutated.

22-25. (canceled)

26. A method of identifying an agent that modulates an activity of CSU52 comprising the steps of:
a) contacting a host cell comprising the nucleic acid of claim 4 that encodes a functional CSU52 polypeptide with a test agent;
b) determining if at least one activity of CSU52 is modulated, such that if an activity of CSU52 is modulated, the test agent is an agent that modulates CSU52 activity.

27. The method of claim 26, wherein the host cell is a Candida albicans cell.

28-30. (canceled)

31. A method of inhibiting Candida albicans cell growth comprising administering to the cell an agent that reduces CSU52 expression.

32. The method of claim 31, wherein the agent is selected from the group consisting of a drug, a small molecule, an antisense oligonucleotide, siRNA, ribozyme or an antibody.

33. The method of claim 31, wherein the cell is in a subject.

34. The method of claim 33, wherein the subject is human.

35. A purified polypeptide comprising an amino acid sequence at least about 95% identical to the sequence of SEQ ID NO: 7.

36. The polypeptide of claim 35 comprising a CSU53 polypeptide comprising SEQ ID NO: 7.

37. (canceled)

38. A nucleic acid encoding the polypeptide of claim 36.

39-45. (canceled)

46. An isolated antibody or fragment thereof that specifically binds the polypeptide of claim 36.

47-54. (canceled)

55. A Candida albicans cell comprising the CSU53 nucleic acid sequence of claim 38 wherein the nucleic acid sequence is mutated.

56. The cell of claim 55, wherein a CSU53 nucleic acid sequence is mutated in one or more CSU53 genes.

57-59. (canceled)

60. A method of identifying an agent that modulates an activity of CSU53 comprising the steps of:
(a) contacting a host cell comprising the nucleic acid of claim 38 that encodes a functional CSU53 polypeptide with a test agent;
(b) determining if at least one activity of CSU53 is modulated, such that if an activity of CSU53 is modulated, the test agent is an agent that modulates CSU53 activity.

61. The method of claim 60, wherein the host cell is a Candida albicans cell.

62-63. (canceled)

64. The method of claim 60, wherein the activity of CSU53 is transcriptional regulation of SOU1.

65. A method of inhibiting Candida albicans cell growth comprising administering to the cell an agent that reduces CSU53 expression.

66. The method of claim 65, wherein the agent is selected from the group consisting of a drug, a small molecule, an antisense oligonucleotide, siRNA, ribozyme or an antibody.

67-68. (canceled)

69. A purified polypeptide comprising an amino acid sequence at least about 95% identical to the sequence of SEQ ID NO: 8.

70. The polypeptide of claim 69 comprising a CSU55 polypeptide comprising SEQ ID NO: 8.

71. (canceled)

72. A nucleic acid encoding the polypeptide of claim 70.

73-79. (canceled)

80. An isolated antibody or fragment thereof that specifically binds the polypeptide of claim 70.

81-88. (canceled)

89. A Candida albicans cell comprising the CSU55 nucleic acid sequence of claim 72 wherein the nucleic acid sequence is mutated.

90-93. (canceled)

94. A method of identifying an agent that modulates an activity of CSU55 comprising the steps of:
(a) contacting a host cell comprising the nucleic acid of claim 72 that encodes a functional CSU55 polypeptide with a test agent;
(b) determining if at least one activity of CSU55 is modulated, such that if an activity of CSU55 is modulated, the test agent is an agent that modulates CSU55 activity.

95. (canceled)

96. The method of claim 94, wherein the agent that modulates an activity of CSU55 increases an activity of CSU55.

97. (canceled)

98. The method of claim 94, wherein the activity of CSU55 is transcriptional regulation of SOU1.

99. A method of inhibiting Candida albicans cell growth comprising administering to the cell an agent that reduces CSU55 expression.

100. The method of claim 99, wherein the agent is selected from the group consisting of a drug, a small molecule, an antisense oligonucleotide, siRNA, ribozyme or an antibody.

101-102. (canceled)

103. A purified polypeptide comprising an amino acid sequence at least about 95% identical to the sequence of SEQ ID NO:1.

104. The polypeptide of claim 103 comprising SEQ ID NO: 1.

105. (canceled)

106. A nucleic acid encoding the polypeptide of claim 104.

107-125. (canceled)

126. A method of reducing CSU51 expression in a cell comprising administering to the cell an antisense oligonucleotide that specifically binds to mRNA transcribed from the CSU51 gene under conditions that allow hybridization, wherein the CSU51 mRNA comprises SEQ ID NO: 2.

127-128. (canceled)

129. A method of identifying an agent that modulates an activity of CSU51 comprising the steps of:
(a) contacting a host cell comprising the nucleic acid of claim 106 that encodes a functional CSU51 polypeptide with a test agent;
(b) determining if at least one activity of CSU51 is modulated, such that if an activity of CSU51 is modulated, the test agent is an agent that modulates CSU51 activity.

130. (canceled)

131. The method of claim 129, wherein the agent that modulates an activity of CSU51 increases an activity of CSU51.

132. (canceled)

133. The method of claim 129, wherein the activity of CSU51 is transcriptional regulation of SOU1.

134. A method of inhibiting Candida albicans cell growth comprising administering to the cell an agent that reduces CSU51 expression.

135. The method of claim 134, wherein the agent is selected from the group consisting of a drug, a small molecule, an antisense oligonucleotide, siRNA, ribozyme or an antibody.

136-137. (canceled)


SEQUENCE LISTING (11 sequences)

SEQ ID NO: 1 - protein, 84 aa, Artificial Sequence
  Description of Artificial Sequence note=synthetic construct
MQFTKVIASLALVASINAKFHNTTGAAAGNGTVAGGSNSTGGAGSHNGTGASNGSSSKSSGSGAAVNSVTGLAALAAVGAALLY

SEQ ID NO: 2 - DNA, 252 bp, Artificial Sequence
  Description of Artificial Sequence note=synthetic construct
atgcaattcaccaaagttatcgcttcattagccttagttgcttccatcaacgctaaattccacaacaccactggtgctgctgctggtaacggtaccgttgctggtggttccaactctactggtggtgctggttcccataacggtactggtgcttctaacggttcttccagtaaatcttctggttccggtgctgctgtcaactccgtcactggtttggctgctttagctgctgttggtgctgctttattgtac

SEQ ID NO: 3 - DNA, 900 bp, Artificial Sequence
  Description of Artificial Sequence note=synthetic construct
tttcaatctgctgctaaattaggaaacatatttaaccagattaggttaaaggattttttttctctctctttctttctatagtctaatcctgtatataagctgtggtgttagaagaagaataataataataataataatcattattattattgttgttgcgtttctttttttttttgtgtgagttacccaccttgtgtacaggaatatgataggaaatttgttagtcaaaaatttcattgatgcatttgtcaccgggtacacgcacacacataatgtaaagataaacaaagcaaagtaaaaaaaaatttttattttcgcccttcaacatagacaaaaaaaatttttactcttttttattaaggcggcaaaaaagaataactttttaccaaaatatataaaaagggttacaattcacccacatgtagtttttttaactctttttccttttccttctttttcctttttttttcttctttttctacaaggataattttaattcaatctttttattccatcaattacacatatatataatcaacaatgcaattcaccaaagttatcgcttcattagccttagttgcttccatcaacgctaaattccacaacaccactggtgctgctgctggtaacggtaccgttgctggtggttccaactctactggtggtgctggttcccataacggtactggtgcttctaacggttcttccagtaaatcttctggttccggtgctgctgtcaactccgtcactggtttggctgctttagctgctgttggtgctgctttattgtactagatgtgcacaacaatacattataagaagaaacaatttattttgttagagaataataataaaagaactgggaaaaaaatgataaaagaaaaagagagagaagccact

SEQ ID NO: 4 - DNA, 21 bp, Artificial Sequence
  Description of Artificial Sequence note=synthetic construct
caattcaccaaagttatcgct

SEQ ID NO: 5 - DNA, 21 bp, Artificial Sequence
  Description of Artificial Sequence note=synthetic construct
gtacaataaagcagcaccaac

SEQ ID NO: 6 - protein, 64 aa, Artificial Sequence
  Description of Artificial Sequence note=synthetic construct
MCIYLLHIVMMLQNFIITVNKFFVFAPPVNLLFCQTGILVFHHHHQTRANNTTILGRDRIITRR

SEQ ID NO: 7 - protein, 303 aa, Artificial Sequence
  Description of Artificial Sequence note=synthetic construct
MSSTQKQKKGAVDFVAGGVAGLFEALCCHPLDTIKVRMQLYKKSGQKPPGFIKTGVNIVQKEGFLSLYKGLGAVVIGIVPKMAIRFSSYEFYRSFFLDENGKISTGKTFLAGVGAGITESVMVVNPMEVVKIRLQAQHHSMKDPLDIPKYRNAPHAAYLIVKEEGFSTLYRGVSLTCARQATNQGANFATYSTIKAYLQKQQNTELLPAWQTSIVGLISGAVGPLTNAPLDTIKTRLQKSKFTNKENGLVRIVKIGKQLVKEEGINALYKGITPRIMRVAPGQAVVFTVYEAVKHYLTNEPTA

SEQ ID NO: 8 - protein, 40 aa, Artificial Sequence
  Description of Artificial Sequence note=synthetic construct
MILCPTSYVWYVKQLVIDNFFEIMYNLVKSWSLIKKKRMK

SEQ ID NO: 9 - DNA, 195 bp, Artificial Sequence
  Description of Artificial Sequence note=synthetic construct
atgtgcatatatttattacatattgtaatgatgttacagaattttattattaccgtgaataaattttttgtttttgcaccccctgtaaatttgttattttgtcaaactggtattcttgtgttccaccaccaccaccagacccgtgctaataatactaccattttagggcgagacagaattataacaagacgataa

SEQ ID NO: 10 - DNA, 912 bp, Artificial Sequence
  Description of Artificial Sequence note=synthetic construct
atgtcttctacacaaaaacaaaagaagggtgctgttgatttcgttgctgggggtgttgctggtttattcgaagctttgtgttgtcatccattagataccatcaaagtgagaatgcaattatacaaaaaatccggtcaaaaaccaccaggttttattaaaacaggtgtcaatattgtccaaaaagaaggatttttgtcattatataaaggtttgggggccgttgttattggtattgtgccaaaaatggctatcagattcagttcatatgaattttaccgttcattctttttagacgaaaatggcaaaatttccactggtaagactttccttgctggtgttggtgctggtattaccgaatctgtcatggttgttaatcctatggaagttgtgaaaattagattacaagcacaacatcattctatgaaggacccattggacattccaaaatacagaaacgctcctcatgctgcatatcttattgtcaaggaagaaggtttcagtactttataccgtggtgtttctttaacttgtgccagacaagctaccaaccaaggtgctaactttgctacatattctaccatcaaagcatatcttcaaaaacaacaaaacactgaattattaccagcatggcaaaccagtattgtcggtttgatttctggtgcagtcggtccattaaccaatgctccattggataccattaaaacaagattacaaaagagtaagtttaccaacaaggaaaacggattggttcgtattgtcaaaatcggtaaacaattagtcaaagaagaaggtattaacgctttgtacaagggtatcactccaagaatcatgagagttgctccaggtcaagctgtggtattcacagtgtatgaagctgtcaaacattatttgacaaatgaacctactgcttaa

SEQ ID NO: 11 - DNA, 123 bp, Artificial Sequence
  Description of Artificial Sequence note=synthetic construct
atgattttatgccctacatcgtacgtttggtacgtgaaacaacttgtaattgataatttttttgaaattatgtataatttagttaaaagttggagtttgatcaagaaaaagcgaatgaagtga